SEQUENCE LISTING (14 sequences)

SEQ ID NO: 1 - DNA, 3393 bp, Homo sapiens
atgttggagatctgcctgaagctggtgggctgcaaatccaagaaggggctgtcctcgtcctccagctgttatctggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatccgctgagcagcgggatcaatggcagcttcttggtgcgtgagagtgagagcagtcctagccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctagtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctagagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgaccgttcccaggtgtatgagctgctagagaaggactaccgcatgaagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgactaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgtctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagcctggaaaggcagtgccttagggacccctgctgcagctgagccagtgacccccaccagcaaagcaggctcaggtgcaccaaggggcaccagcaagggccccgccgaggagtccagagtgaggaggcacaagcactcctctgagtcgccagggagggacaaggggaaattgtccaagctcaaacctgccccgccgcccccaccagcagcctctgcagggaaggctggaggaaagccctcgcagaggcccggccaggaggctgccggggaggcagtcttgggcgcaaagacaaaagccacgagtctggttgatgctgtgaacagtgacgctgccaagcccagccagccggcagagggcctcaaaaagcccgtgctcccggccactccaaagccacaccccgccaagccgtcggggacccccatcagcccagcccccgttcccctttccacgttgccatcagcatcctcggccttggcaggggaccagccgtcttccactgccttcatccctctcatatcaacccgagtgtctcttcggaaaacccgccagcctccagagcgggccagcggcgccatcaccaagggcgtggtcttggacagcaccgaggcgctgtgcctcgccatctctgggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgtcagcaggcagtggtccggcggccactcaggacttcagcaagctcctcagttcggtgaaggaaatcagtgacatagtgcagaggtag

SEQ ID NO: 2 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYPLSSGINGSFLVRESESSPSQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPSFAEIHQAFETMFQESSISDEVEKELGKQGVRGAVTTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSVSCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAWKGSALGTPAAAEPVTPTSKAGSGAPRGTSKGPAEESRVRRHKHSSESPGRDKGKLSKLKPAPPPPPAASAGKAGGKPSQRPGQEAAGEAVLGAKTKATSLVDAVNSDAAKPSQPAEGLKKPVLPATPKPHPAKPSGTPISPAPVPLSTLPSASSALAGDQPSSTAFIPLISTRVSLRKTRQPPERASGAITKGVVLDSTEALCLAISGNSEQMASHSAVLEAGKNLYTFCVSYVDSIQQMRNKFAFREAINKLENNLRELQICPASAGSGPAATQDFSKLLSSVKEISDIVQR

SEQ ID NO: 3 - DNA, 22 bp, Artificial
  Synthesized oligonucleotide.
gaagcttctccctgacatccgt

SEQ ID NO: 4 - DNA, 22 bp, Artificial
  Synthesized oligonucleotide.
gccaggctctcgggtgcagtcc

SEQ ID NO: 5 - DNA, 24 bp, Artificial
  Synthesized oligonucleotide.
gcgcaacaagcccactgtctatgg

SEQ ID NO: 6 - DNA, 24 bp, Bacteriophage M13
cgccagggttttcccagtcacgac

SEQ ID NO: 7 - DNA, 24 bp, Bacteriophage M13
agcggataacaatttcacacagga

SEQ ID NO: 8 - DNA, 20 bp, Artificial
  Synthesized oligonucleotide.
gcagagtcagaatccttcag

SEQ ID NO: 9 - DNA, 20 bp, Artificial
  Synthesized oligonucleotide.
tttgtaaaaggctgcccggc

SEQ ID NO: 10 - DNA, 30 bp, Artificial
  Synthesized oligonucleotide.
cggcattgcgggacacaggcccatggtacc

SEQ ID NO: 11 - DNA, 22 bp, Artificial
  Synthesized oligonucleotide.
tgaccaactcgtgtgtgaaact

SEQ ID NO: 12 - DNA, 34 bp, Artificial
  Synthesized oligonucleotide.
ggggaattcgccaccatgttggagatctgcctga

SEQ ID NO: 13 - DNA, 20 bp, Artificial
  Synthesized oligonucleotide.
ttcaccaccatggagaaggc

SEQ ID NO: 14 - DNA, 20 bp, Artificial
  Synthesized oligonucleotide.
caggaaatgagcttgacaaa

(19) United States
(12) Patent Application Publication
(10) Pub. No.: US 2009/0163510 A1
(43) Pub. Date: Jun. 25, 2009

(54) Methods of Identifying and Treating Individuals Exhibiting Mutant SRC Kinase Polypeptides

(75) Inventor: Francis Y. Lee, Yardley, PA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/988,778

(22) PCT Filed: Jul. 13, 2006
(86) PCT No.: PCT/US2006/027460

§ 371 Date: Jan. 14, 2008

Related U.S. Application Data
(60) Provisional application No. 60/699,675, filed on Jul. 15, 2005.

(51) Int. Cl. A61K 31/506; C12Q 1/48

(52) U.S. Cl. 514/252.19; 435/15

(57) ABSTRACT

The invention described herein relates to mutant SRC kinase proteins, and to diagnostic and therapeutic methods and compositions useful in the management of disorders, for example cancers, involving cells that express such mutant SRC kinase proteins.

This application claims benefit to provisional application U.S. Ser. No. 60/699,675 filed Jul. 15, 2005, under 35 U.S.C. 119(e). The entire teachings of the referenced applications are incorporated herein by reference.

### FIELD OF THE INVENTION

The invention described herein relates to mutant SRC kinase proteins, and to diagnostic and therapeutic methods and compositions useful in the management of disorders, for example cancers, involving cells that express such mutant SRC kinase proteins.

### BACKGROUND OF THE INVENTION

Cancer is the second leading cause of human death next to coronary disease. Worldwide, millions of people die from cancer every year. In the United States alone, cancer causes the death of well over a half-million people annually, with some 1.4 million new cases diagnosed per year. While deaths from heart disease have been declining significantly, those resulting from cancer generally are on the rise and are predicted to become the leading cause of death in the developed world.

Chronic Myelogenous Leukemia (CML) is a myeloproliferative disorder that is characterized by Philadelphia chromosome translocation. (see, e.g. C. L. Sawyers, N. En. J. Med. 340, 1330 (1999); and S. Faderl et al., N. Engl. J. Med. 341,164 (1999)). A reciprocal translocation between chromosomes 9 and 22 produces the oncogenic BCR-ABL fusion protein. The BCL-ABL protein constitutes tyrosine kinase activity and is known to produce CML-like disease in mice (see, e.g. J. B. Konopka et al., Proc. Natl. Acad. Sci. U.S. 82, 1810 (1985); G. Q. Daley et al., Science 247, 824 (1990); and N. Heisterkamp et al., Nature 344, 251 (1990)). In fact, 95% of CML is Philadelphia-positive (Ph+). A single mutation on BCR-ABL is sufficient for the incidence of CML disease (D. G. Savage, K. H. Antman, NEJM 346(9) (2002).

CML progresses through distinct clinical stages. The earliest stage, termed chronic phase, is characterized by expansion of terminally differentiated neutrophils. Over several years the disease progresses to an acute phase termed blast crisis, characterized by maturation arrest with excessive numbers of undifferentiated myeloid or lymphoid progenitor cells. The BCR-ABL oncogene is expressed at all stages, but blast crisis is characterized by multiple additional genetic and molecular changes.

Imatinib mesylate (also known as STI-571) is a potent BCR-ABL tyrosine kinase inhibitor and is now standard of care in CML patients. As used herein the term “imatinib” is used to refer to imatinib mesylate or STI-571. Although imatinib is a potent inhibitor of the kinase activity of wild type BCR-ABL, many mutant BCR-ABL isoforms are resistant to clinically achievable doses of imatinib. Clinical resistance is primarily mediated by mutations within the kinase domain of BCR-ABL and, to a lesser extent, by amplification of the BCR-ABL genomic locus (M. E. Gorre et al., Science 193, 876 (2001)). Imatinib can bind to the adenosine triphosphate (ATP)-binding site of ABL only when its activation loop is “closed” and thus the protein is in inactive conformation. This conformation-specific requirement contributes to imatinib's selectivity and the resistance shown in CML patients.

Mutations in the ABL kinase domain have been associated with imatinib resistance in CML patient (N. von Bubnoff, F. Schneller, C. Peschel, J. Duyster, Lancet 359, 487 (2002) and S. Branford et al., Blood 99, 3472 (2002)). To date, 17 different amino acid positions within the ABL kinase domain were identified. The majority of amino acid substitutions are believed to cause resistance by impairing the ability of the kinase domain flexibility, such that the BCR-ABL kinase domain is unable to assume the optimal inactive conformation required for imatinib binding (N. P. Shah et al., Cancer Cell 2, 117 (2002); which is hereby incorporated herein by reference).

The single letter amino acid sequence of wild-type human BCR-ABL protein shown in FIG. 1 (SEQ ID NO:2; gi/77942; and gi|M14752.1). The mutations are shown in FIG. 2. The nucleic acid sequence of BCR-ABL is encoded by nucleotides 1 to 3681 of SEQ ID NO:1 (gi|177942; and gi|M14752.1).

′N-(2-chloro-6-methylphenyl)-2-(6-(4-(2-hydroxyethyl)piperazin-1-yl)-2-methylpyrimidin-4-ylamino)thiazole-5-carboxamide is a synthetic small-molecule inhibitor of several SRC-family kinases, including BCR-ABL. Structural studies indicate that protein tyrosine kinase inhibitors, including ′N-(2-chloro-6-methylphenyl)-2-[[6-[4-(2-hydroxyethyl)-1-piperazinyl]-2-methyl-4-pyrimidinyl]amino]-5-thiazolecarboxamide, bind to the ATP-binding site in ABL, but without regard for the position of the active loop, which can be in the active or inactive conformation (B. Nagar et al., Cancer Res. 62, 4236 (2002)). The less stringent conformation requirement for binding to the ABL kinase domain is one reason ′N-(2-chloro-6-methylphenyl)-2-[[6-[4-(2-hydroxyethyl)-1-piperazinyl]-2-methyl-4-pyrimidinyl]amino]-5-thiazolecarboxamide retains activity against many imatinib-resistant mutants. ′N-(2-chloro-6-methylphenyl)-2-[[6-[4-(2-hydroxyethyl)-1-piperazinyl]-2-methyl-4-pyrimidinyl]amino]-5-thiazolecarboxamide was more potent than imatinib at inhibiting nonmutated BCR-ABL (BCR-ABL/WT) kinase activity in a cell-based assay. In addition, the kinase activity of 14 out of 15 different clinically relevant, imatinib-resistant BCR-ABL isoforms was successfully inhibited in the low nanomolar range (N. P. Shah et al., Science 305, 399 (2003)). Within the 1 to 10 nM range, there are subtle yet highly reproducible differences in the sensitivity of certain BCR-ABL mutant isoforms. For example, three- to five-fold higher concentrations of ′N-(2-chloro-6-methylphenyl)-2-[[6-[4-(2-hydroxyethyl)-1-piperazinyl]-2-methyl-4-pyrimidinyl]amino]-5-thiazolecarboxamide were required to inhibit the growth of Ba/F3 cells (a murine pro-B-cell line) expressing the F317L mutant, whereas the Q252R mutant was consistently more sensitive than non-mutated BCR-ABL.

In view of the imatinib resistance observed in certain cancers with cells containing certain BCR-ABL mutant isoforms, there is a need for diagnostic and therapeutic procedures and compositions tailored to address this condition. Particularly there is a need for a treatment for cancer, mastocytosis and related disorders involving mutant BCR-ABL kinase. The invention provided herein satisfies this need.

### SUMMARY OF THE INVENTION

imatinib is a small-molecule inhibitor of the BCR/ABL tyrosine kinase that produces clinical remissions in CML patients with minimal toxicity relative to older treatment modalities. imatinib is now frontline therapy for CML but resistance is increasingly encountered. According to one study, the estimated 2-year incidence of resistance to imatinib mesylate was 80% in blastic phase, 40% to 50% in accelerated phase, and 10% in chronic phase post-interferon-x failure (Kantarjian et al, Blood, 101(2):473-475 (2003). ′N-(2-chloro-6-methylphenyl)-2-[[6-[4-(2-hydroxyethyl)-1-piperazinyl]-2-methyl-4-pyrimidinyl]amino]-5-thiazolecarboxamide is an ATP-competitive, dual SRC/ABL inhibitor (Lombardo, L. J., et al., J. Med. Chem., 47:6658-6661 (2004)). Notably, ′N-(2-chloro-6-methylphenyl)-2-[[6-[4-(2-hydroxyethyl)-1-piperazinyl]-2-methyl-4-pyrimidinyl]amino]-5-thiazolecarboxamide has been shown to inhibit BCR-ABL imatinib-resistant mutations that are found in some CML patients with acquired clinical resistance to imatinib. On account of the demonstration that patients harboring different Src mutations, particularly BCR/ABL mutations, have varying degrees of resistance and/or sensitivity to imatinib and ′N-(2-chloro-6-methylphenyl)-2-[[6-[4-(2-hydroxyethyl)-1-piperazinyl]-2-methyl-4-pyrimidinyl]amino]-5-thiazolecarboxamide, respectively, the inventors of the present invention describe for the first time methods to identify patients who may most benefit from the combination of ′N-(2-chloro-6-methylphenyl)-2-[[6-[4-(2-hydroxyethyl)-1-piperazinyl]-2-methyl-4-pyrimidinyl]amino]-5-thiazolecarboxamide with other protein tyrosine kinase inhibitors, or other agents.

The structure and use of ′N-(2-chloro-6-methylphenyl)-2-[[6-[4-(2-hydroxyethyl)-1-piperazinyl]-2-methyl-4-pyrimidinyl]amino]-5-thiazolecarboxamide as an anticancer agent is described in Lombardo, L. J., et al., J. Med. Chem., 47:6658-6661 (2004) and is described in the following US patents and pending applications, incorporated herein by reference: U.S. Pat. No. 6,596,746, granted Jul. 22, 2003; U.S. Ser. No. 10/395,503, filed Mar. 24, 2003.

The structure and use of imatinib as an anticancer agent is described in B. J. Druker et al., N. Engl. J. Med. 344, 1031 (2001) and S. G. O'Brien et al., N. Engl. J. Med. 348, 994 (2003).

As described above, certain mutations in the BCR/ABL kinase cause it to be resistant to the therapeutic effects of imatinib. Identification of patients harboring cancer cells that contain such an imatinib-resistant BCR/ABL mutation(s) and the treatment of such patients, with a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide or a dosing regimen of ′N-(2-chloro-6-methylphenyl)-2-[[6-[4-(2-hydroxyethyl)-1-piperazinyl]-2-methyl-4-pyrimidinyl]amino]-5-thiazolecarboxamide, that can inhibit proliferation and/or induce apoptosis of is an object of the present invention. Provided herein is such a method, useful in inhibiting proliferation and/or inducing apoptosis of cell lines that are resistant, or at least partially imatinib resistant, or at least partially resistant to a protein tyrosine kinase inhibitor, to treatment with imatinib in patients harboring such cells.

Therefore, the present invention provides compositions, kits and methods for diagnosing and treating a host, preferably human, having or predisposed to a disease associated with abnormal activity of one or more protein tyrosine kinases. Specifically, the invention provides methods of identifying a mutant BCR/ABL kinase in a host having a disease associated with abnormal activity of said BCR/ABL mutant kinase, and tailoring treatment of said host based upon identification of said mutant BCR/ABL kinase.

The present invention provides kits for screening and diagnosing disorders associated with aberrant or uncontrolled cellular development and with the expression of a SRC kinase mutant as described herein.

The present invention provides a method of screening a biological sample, for example cells that do not respond, or that have stopped responding, or that have a diminished response, to protein tyrosine kinase inhibitors used to inhibit proliferation of said cells. For example, the present invention provides a method of screening cells from an individual suffering from cancer who is being treated with imatinib, and whose cells do not respond or have stopped responding or that have a diminished response to imatinib, for the presence of SRC mutations described herein. The present invention provides certain SRC mutations that, if present, provide the basis upon which to alter treatment of such individual to inhibit proliferation of said cells.

The present invention provides a method of screening cells that do not respond, or that have stopped responding or that have a diminished response, to kinase inhibitors used to induce apoptosis of said cells. For example, the present invention provides a method of screening cells from an individual suffering from cancer who is being treated with imatinib, and whose cells do not respond or have stopped responding or that have a diminished response to imatinib, for the presence of SRC kinase mutations described herein. The present invention provides certain SRC kinase mutations that, if present, provide the basis upon which to alter treatment of such individual to induce apoptosis of said cells.

Also provided is a method of treating a SRC associated disorder, particularly a mutant SRC-associated disorder, comprising obtaining a sample of cells from a patient suffering from said disorder, assaying the cells for the presence of a SRC mutation, such as one or more of those described herein, and treating said patient with a combination or treatment regimen to inhibit proliferation and/or induce apoptosis of said cells.

The invention encompasses a method of treating an individual suffering from cancer, wherein the method comprises assaying cells from said individual to determine the presence of at least one mutation in a SRC kinase protein in said cells, wherein said at least one mutation in a SRC kinase results in said SRC kinase being constitutively activated, and thereby administering to said individual a therapeutically effective amount of a member of the group consisting of: a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib; a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., Compound II); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor, especially a BCR-ABL inhibitor such as imatinib as indicated herein, or AMN107; an increased dosing frequency regimen of ′N-(2-chloro-6-methylphenyl)-2-[[6-[4-(2-hydroxyethyl)-1-piperazinyl]-2-methyl-4-pyrimidinyl]amino]-5-thiazolecarboxamide; and any other combination or dosing regimen comprising ′N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein.

The invention further encompasses a method of treating an individual suffering from cancer, wherein the method comprises assaying cells from said individual to determine the presence of at least one mutation in a SRC kinase protein in said cells, wherein said at least one mutation is selected from the group consisting of M244V, G250E, Q252H, Q252R, Y253F, Y253H, E255K, E255V, T315I, F317L, M351T, E355G, F359V H396R, and F486S of SEQ ID NO:2, and, if said mutation in said Src protein kinase is identified, administering to said individual a therapeutically effective amount of a member of the group consisting of: a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib; a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., COMPOUND II); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; an increased dosing frequency regimen of ′N-(2-chloro-6-methylphenyl)-2-[[6-[4-(2-hydroxyethyl)-1-piperazinyl]-2-methyl-4-pyrimidinyl]amino]-5-thiazolecarboxamide; and any other combination or dosing regimen comprising ′N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein.

The invention further comprises a method of treating an individual suffering from cancer (especially a SRC kinase associated cancer), wherein said individual is or has received administration of a first kinase inhibitor to which the cancer cells in said individual have become resistant or at least partially resistant, comprising assaying cells from said individual to determine the presence of at least one mutation in SRC kinase protein in said cells, wherein said at least one mutation in a SRC kinase results in said cancer cells being resistant or at least partially resistant to said first kinase inhibitor, and, if at least one mutation is present in said SRC kinase protein, administering a therapeutically effective amount of a member of the group consisting of: a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib alone; a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and said first kinase inhibitor; a combination of ′N-(2-chloro-6-methylphenyl)-2-[[6-[4-(21-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., COMPOUND II); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; an increased dosing frequency regimen of ′N-(2-chloro-6-methylphenyl)-2-[[6-[4-(2-hydroxyethyl)-1-piperazinyl]-2-methyl-4-pyrimidinyl]amino]-5-thiazolecarboxamide; and any other combination or dosing regimen comprising ′N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein.

The invention further comprises a method of treating an individual suffering from cancer (especially a SRC associated cancer), wherein said individual is or has received administration of a BCR-ABL inhibitor such as imatinib to which the cancer cells in said individual have become resistant or at least partially resistant, comprising assaying cells from said individual to determine the presence of at least one mutation in a SRC kinase protein in said cells, wherein said at least one mutation in a SRC kinase results in said cancer cells being resistant or at least partially resistant to imatinib, and, if at least one mutation is present in said SRC kinase protein, administering a therapeutically effective amount of a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and another BCR-ABL inhibitor such as imatinib alone or in combination with other agents including, but not limited to Taxol or other protein tyrosine kinase inhibitors. Combinations comprising ′N-(2-chloro-6-methylphenyl)-2-[[6-[4-(2-hydroxyethyl)-1-piperazinyl]-2-methyl-4-pyrimidinyl]amino]-5-thiazolecarboxamide that may be useful to practice the methods of the present invention are described in U.S. Ser. No. 10/886,955, filed Jul. 8, 2004, U.S. Ser. No. 11/265,843, filed Nov. 3, 2005, and U.S. Ser. No. 11/418,338, filed May 4, 2006, each of which are incorporated herein by reference.

The invention further comprises a method of treating an individual suffering from cancer, wherein said individual is or has received administration of imatinib to which the cancer cells in said individual have become resistant or at least partially resistant, comprising assaying cells from said individual to determine the presence of at least one mutation in a SRC kinase protein in said cells, wherein said at least one mutation in a SRC kinase results in said cancer cells being resistant to imatinib or at least partially resistant, and, if at least one mutation is present in said SRC kinase protein, administering a therapeutically effective amount of a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib, including an increased or decreased dose, alone or in combination with other agents including, but not limited to Taxol or other protein tyrosine kinase inhibitors. Combinations comprising a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib that may be useful to practice the methods of the present invention are described in U.S. Ser. No. 10/886,955, filed Jul. 8, 2004, U.S. Ser. No. 11/265,843, filed Nov. 3, 2005, and U.S. Ser. No. 11/418,338, filed May 4, 2006, each of which are incorporated herein by reference.

The invention further comprises a method of treating an individual suffering from cancer (especially a SRC associated cancer), wherein said individual is or has received administration of a first kinase inhibitor to which the cancer cells in said individual have become resistant or at least partially resistant, comprising assaying cells from said individual to determine the presence of at least one mutation in a SRC kinase protein in said cells, wherein said at least one mutation is selected from the group consisting of M244V, G250E, Q252H, Q252R, Y253F, Y253H, E255K, E255V, T315I, F317L, M351T, E355G, F359V H396R, and F486S of SEQ ID NO:2, and, if said mutation is present, administering a therapeutically effective amount of a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib.

The invention further comprises a method of treating an individual suffering from cancer (especially a SRC associated cancer), wherein said individual is or has received administration of imatinib to which the cancer cells in said individual have become resistant or at least partially resistant, comprising assaying cells from said individual to determine the presence of at least one mutation in a SRC kinase protein in said cells, wherein said at least one mutation is selected from the group consisting of M244V, G250E, Q252H, Q252R, Y253F, Y253H, E255K, E255V, T315I, F317L, M351T, E355G, F359V H396R, and F486S of SEQ ID NO:2, and, if said mutation is present, administering a therapeutically effective amount of a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib.

The invention further comprises a method for determining the responsiveness of an individual with a protein tyrosine kinase-associated disorder to treatment with ′N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt or hydrate thereof, in combination with a tubulin stabilizing agent (e.g., pacitaxol, epothilone, taxane, etc.); in combination with a farnysyl transferase inhibitor (e.g., COMPOUND II); in combination with another protein tyrosine kinase inhibitor, for example, an inhibitor of BCR-ABL, such as imatinib and/or AMN-107, wherein said individual has either been previously treated with and developed at least partial resistance to a first kinase inhibitor, or is naïve to treatment with kinase inhibitors, comprising: (a) providing a biological sample from said individual; (b) screening said biological sample for the presence of at least one mutation in a BCR/ABL kinase sequence; and (c) assigning the individual to a positive responder group if a mutant BCR/ABL kinase is identified.

The invention further comprises responsiveness methods wherein said mutant BCR/ABL kinase comprises an amino acid mutation that results in said BCR/ABL kinase being constitutively active.

The invention further comprises responsiveness methods wherein said protein tyrosine kinase-associated disorder is selected from the group consisting of chronic myeloid leukemia (CML), Ph+ ALL, and solid tumors.

The invention further comprises responsiveness methods wherein said mutant BCR/ABL kinase comprises an imatinib resistant BCR/ABL mutation.

The invention further comprises responsiveness methods wherein said imatinib resistant BCR/ABL mutation comprises a mutation at amino acid positions 244, 250, 252, 253, 255, 315, 317, 351, 355, 359, 396 and 486 of SEQ ID NO:2.

The invention further comprises responsiveness methods wherein said mutation is selected from the group consisting of M244V, G250E, Q252H, Q252R, Y253F, Y253H, E255K, E255V, T315I, F317L, M351T, E355G, F359V H396R, and F486S of SEQ ID NO:2.

The invention further comprises responsiveness methods wherein said biological sample is selected from the group consisting of a tissue biopsy, blood, serum, plasma, lymph, ascitic fluid, cystic fluid, urine, sputum, stool, saliva, bronchial aspirate, spinal fluid and hair.

The invention further comprises responsiveness methods wherein said biological sample is a tissue biopsy cell sample or cells cultured therefrom.

The invention further comprises responsiveness methods wherein said biological sample comprises blood cells.

The invention further comprises responsiveness methods of claim 7 wherein said biological sample comprises cells removed from a solid tumor.

The invention further comprises responsiveness methods wherein said biological sample comprises a lysate of said cell sample.

The invention comprises methods of treating an individual suffering from a protein tyrosine kinase associated disorder comprising: (a) providing a biological sample from said individual; (b) assaying said biological sample for the presence of a mutant BCR/ABL kinase; and if a mutant BCR/ABL kinase is identified (c) administering a therapeutically effective amount of ′N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt or hydrate thereof, in combination with imatinib; a higher dose or dosing frequency of said thiazolecarboxamide or a pharmaceutically acceptable salt or hydrate thereof; or combinations of said thiazolecarboxamide or a pharmaceutically acceptable salt or hydrate thereof with a tubulin stabilizing agent, a farnysyl transferase inhibitor, a BCR/ABL T315I inhibitor and/or another protein tyrosine kinase inhibitor; or both a higher dose or dosing frequency of said thiazolecarboxamide or a pharmaceutically.

The invention comprises methods of treatment wherein said mutant BCR/ABL kinase comprises a mutation at amino acid positions 244, 250, 252, 253, 255, 315, 317, 351, 355, 359, 396 and 486 of BCR/ABL kinase (SEQ ID NO:2).

The invention comprises methods of treatment wherein said mutation is selected from the group consisting of M244V, G250E, Q252H, Q252R, Y253F, Y253H, E255K, E255V, T315I, F317L, M351T, E355G, F359V H396R, and F486S of SEQ ID NO:2.

The invention comprises methods of treating an individual suffering from a protein tyrosine kinase associated disorder comprising: (a) providing a biological sample from said individual; (b) assaying said biological sample for at least partial resistance to a first kinase inhibitor; (c) assaying said biological sample for the presence of a mutant BCR/ABL kinase; and if said biological sample is determined to be at least partially resistant to said first kinase inhibitor and contains a mutant BCR/ABL kinase, then (d) administering a therapeutically effective amount of ′N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt or hydrate thereof; a higher dose or dosing frequency of said thiazolecarboxamide or a pharmaceutically acceptable salt or hydrate thereof; or combinations of said thiazolecarboxamide or a pharmaceutically acceptable salt or hydrate thereof with a tubulin stabilizing agent, a farnysyl transferase inhibitor, a BCR/ABL T315I inhibitor and/or another protein tyrosine kinase inhibitor; or both a higher dose or dosing frequency of said thiazolecarboxamide or a pharmaceutically.

The invention comprises methods of treatment wherein said first kinase inhibitor is a BCR/ABL kinase inhibitor.

The invention comprises methods of treatment wherein said mutant BCR/ABL kinase comprises a mutation at amino acid positions 244, 250, 252, 253, 255, 315, 317, 351, 355, 359, 396 and 486 of SEQ ID NO:2.

The invention comprises methods of treatment wherein said mutation is selected from the group consisting of M244V, G250E, Q252H, Q252R, Y253F, Y253H, E255K, E255V, T315L, F317L, M351T, E355G, F359V H396R, and F486S of SEQ ID NO:2.

The invention comprises methods of treatment wherein said first kinase inhibitor comprises imatinib.

The invention comprises methods of establishing a treatment regimen for an individual suffering from a protein tyrosine kinase associated disorder comprising: (a) providing a biological sample from said individual; (b) screening said biological sample for the presence of at least one mutation in a BCR/ABL kinase sequence; and, if at least one mutation in a BCR/ABL kinase sequence is present in said biological sample, (c) administering to said individual as part of a treatment regimen a pharmaceutical composition comprising ′N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt or hydrate thereof; a higher dose or dosing frequency of said thiazolecarboxamide or a pharmaceutically acceptable salt or hydrate thereof; or combinations of said thiazolecarboxamide or a pharmaceutically acceptable salt or hydrate thereof with a tubulin stabilizing agent, a farnysyl transferase inhibitor, a BCR/ABL T315I inhibitor and/or another protein tyrosine kinase inhibitor; or both a higher dose or dosing frequency of said thiazolecarboxamide or a pharmaceutically.

The invention comprises methods of establishing a treatment regimens wherein said at least on mutation comprises a mutation at amino acid positions 244, 250, 252, 253, 255, 315, 317, 351, 355, 359, 396 and 486 of SEQ ID NO:2.

The invention comprises methods of establishing a treatment regimen wherein said pharmaceutical composition comprises at least one additional kinase inhibitor.

The invention comprises methods of establishing a treatment regimen wherein said mutation is selected from the group consisting of M244V, G250E, Q252H, Q252R, Y253F, Y253H, E255K, E255V, T315L, F317L, M351T, E355G, F359V H396R, and F486S.

The invention comprises methods of establishing a treatment regimen wherein said treatment regimen comprises a therapeutically effective amount of ′N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt or hydrate thereof; a higher dose or dosing frequency of said thiazolecarboxamide or a pharmaceutically acceptable salt or hydrate thereof; or combinations of said thiazolecarboxamide or a pharmaceutically acceptable salt or hydrate thereof with a tubulin stabilizing agent, a farnysyl transferase inhibitor, a BCR/ABL T315I inhibitor and/or another protein tyrosine kinase inhibitor; or both a higher dose or dosing frequency of said thiazolecarboxamide or a pharmaceutically.

The invention comprises methods of establishing a treatment regimen wherein said biological sample is selected from the group consisting of a tissue biopsy, blood, serum, plasma, lymph, ascitic fluid, cystic fluid, urine, sputum, stool, saliva, bronchial aspirate, spinal fluid and hair.

The invention comprises methods of establishing a treatment regimen wherein said biological sample is a tissue biopsy cell sample or cells cultured therefrom.

The invention comprises methods of establishing a treatment regimen wherein said biological sample comprised blood cells.

The invention comprises methods of establishing a treatment regimen wherein said biological sample comprises cells removed from a solid tumor.

The invention comprises methods of establishing a treatment regimen wherein said biological sample comprises a lysate of said cell sample.

The invention comprises methods of establishing a treatment regimen for an individual suffering from a protein tyrosine kinase associated disorder comprising: (a) providing a biological sample from said individual; (b) assaying said biological sample for at least partial resistance to a first kinase inhibitor; (c) assaying said biological sample for the presence of at least one mutation in a BCR/ABL kinase sequence; and, if said biological sample is determined to be at least partially resistant to said first kinase inhibitor and contain a mutant BCR/ABL kinase, then (d) administering to said individual as part of a treatment regimen a pharmaceutical composition comprising ′N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt thereof; a higher dose or dosing frequency of said thiazolecarboxamide or a pharmaceutically acceptable salt or hydrate thereof; or combinations of said thiazolecarboxamide or a pharmaceutically acceptable salt or hydrate thereof with a tubulin stabilizing agent, a farnysyl transferase inhibitor, a BCR/ABL T315I inhibitor and/or another protein tyrosine kinase inhibitor; or both a higher dose or dosing frequency of said thiazolecarboxamide or a pharmaceutically.

The invention comprises methods of establishing a treatment regimen wherein said at least on mutation comprises a mutation at amino acid positions 244, 250, 252, 253, 255, 315, 317, 351, 355, 359, 396 and 486 of SEQ ID NO:2.

The invention comprises methods of establishing a treatment regimen wherein said treatment regimen further comprises administration of at least one additional protein tyrosine kinase inhibitor.

The invention comprises methods of establishing a treatment regimen wherein said mutation is selected from the group consisting of M244V, G250E, Q252H, Q252R, Y253F, Y253H, E255K, E255V, T315I, F317L, M351T, E355G, F359V H396R, and F486S of SEQ ID NO:2.

The invention comprises methods of treating an individual suffering from a mutant BCR/ABL kinase associated disorder comprising: (a) providing a biological sample from said individual; (b) assaying said biological sample for the presence of a mutant BCR/ABL kinase, wherein said mutant BCR/ABL kinase is constitutively active; and, if a constitutively active mutant BCR/ABL kinase is present in said sample, then (c) administering to said individual a pharmaceutical composition comprising ′N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt thereof; a higher dose or dosing frequency of said thiazolecarboxamide or a pharmaceutically acceptable salt or hydrate thereof; or combinations of said thiazolecarboxamide or a pharmaceutically acceptable salt or hydrate thereof with a tubulin stabilizing agent, a farnysyl transferase inhibitor, a BCR/ABL T315I inhibitor and/or another protein tyrosine kinase inhibitor; or both a higher dose or dosing frequency of said thiazolecarboxamide or a pharmaceutically.

The invention comprises methods of treating an individual suffering from a mutant BCR/ABL kinase associated disorder comprising administering a therapeutically effective amount of ′N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt thereof; a higher dose or dosing frequency of said thiazolecarboxamide or a pharmaceutically acceptable salt or hydrate thereof; or combinations of said thiazolecarboxamide or a pharmaceutically acceptable salt or hydrate thereof with a tubulin stabilizing agent, a farnysyl transferase inhibitor, a BCR/ABL T315I inhibitor and/or another protein tyrosine kinase inhibitor; or both a higher dose or dosing frequency of said thiazolecarboxamide or a pharmaceutically.

The invention comprises a kit for use in determining treatment strategy for an individual with a protein tyrosine kinase-associated disorder, comprising: (a) a means for detecting a mutant BCR/ABL kinase in a biological sample from said patient; and optionally (b) instructions for use and interpretation of the kit results.

The invention comprises kits wherein said kit comprises said instructions and wherein said treatment strategy comprises administration of a therapeutically effective amount of ′N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt thereof; a higher dose or dosing frequency of said thiazolecarboxamide or a pharmaceutically acceptable salt or hydrate thereof; or combinations of said thiazolecarboxamide or a pharmaceutically acceptable salt or hydrate thereof with a tubulin stabilizing agent, a farnysyl transferase inhibitor, a BCR/ABL T315I inhibitor and/or another protein tyrosine kinase inhibitor; or both a higher dose or dosing frequency of said thiazolecarboxamide or a pharmaceutically.

The invention comprises kits wherein said mutant BCR/ABL kinase comprises mutations at positions 244, 250, 252, 253, 255, 315, 317, 351, 355, 359, 396 and 486 of SEQ ID NO:2.

The invention comprises kits wherein said mutation is selected from the group consisting of M244V, G250E, Q252H, Q252R, Y253F, Y253H, E255K, E255V, T315I, F317L, M351T, E355G, F359V H396R, and F486S of SEQ ID NO:2.

The invention comprises kits further comprising a means for obtaining a biological sample from said individual.

The invention comprises kits for use in treating an individual with a mutant BCR/ABL kinase associated disorder, comprising: (a) a means for detecting mutations at amino acid positions 244, 250, 252, 253, 255, 315, 317, 351, 355, 359, 396 and 486 of a BCR/ABL kinase from a biological sample from said individual; (b) a therapeutically effective amount of ′N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, in combination with a tubulin stabilizing agent (e.g., pacitaxol, epothilone, taxane, etc.); a higher dose or dosing frequency of said thiazolecarboxamide or a pharmaceutically acceptable salt or hydrate thereof; or combinations of said thiazolecarboxamide or a pharmaceutically acceptable salt or hydrate thereof with a tubulin stabilizing agent, a farnysyl transferase inhibitor, a BCR/ABL T315I inhibitor and/or another protein tyrosine kinase inhibitor; or both a higher dose or dosing frequency of said thiazolecarboxamide or a pharmaceutically.

The invention comprises kits wherein said mutation is selected from the group consisting of M244V, G250E, Q252H, Q252R, Y253F, Y253H, E255K, E255V, T315I, F317L, M351T, E355G, F359V H396R, and F486S of SEQ ID NO:2.

The invention comprises kits further comprising a means for obtaining a biological sample from said individual.

### BRIEF DESCRIPTION OF THE FIGURES

FIGS. 1A-E show the polynucleotide sequence (SEQ ID NO:1) and deduced amino acid sequence (SEQ ID NO:2) of the wild-type BCR-ABL polypeptide. The standard one-letter abbreviation for amino acids is used to illustrate the deduced amino acid sequence. The polynucleotide sequence contains a sequence of 3393 nucleotides (SEQ ID NO:1; gi|177942; and gi|M14752.1), encoding a polypeptide of 1130 amino acids (SEQ ID NO:2; gi|177942; and gi|M14752.1).

FIG. 2 provides a summary of the Src kinase mutations of the amino acid sequence of SEQ ID NO:2 useful for the diagnostic methods of the present invention.

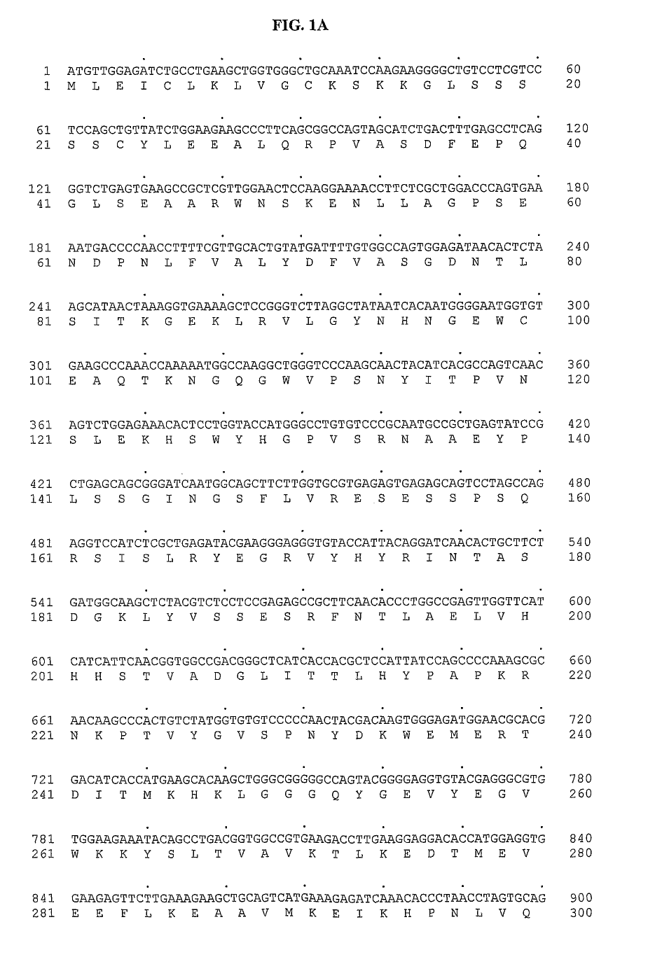

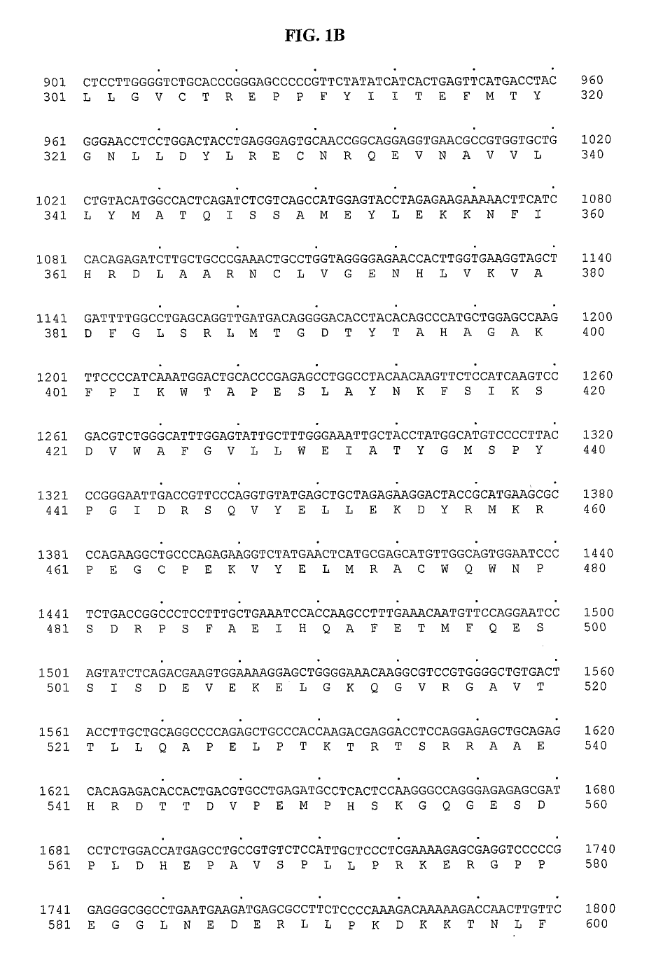

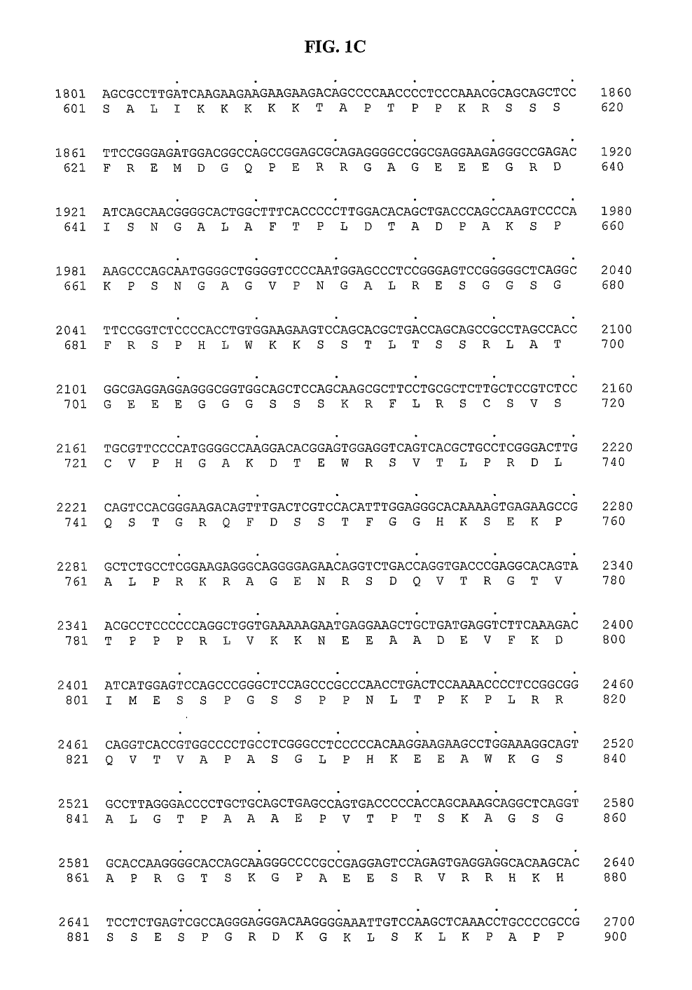

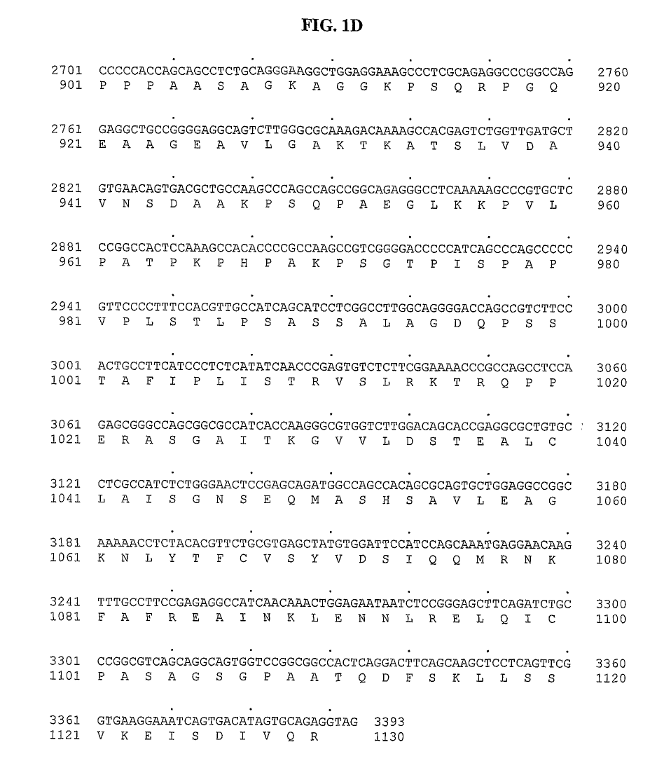

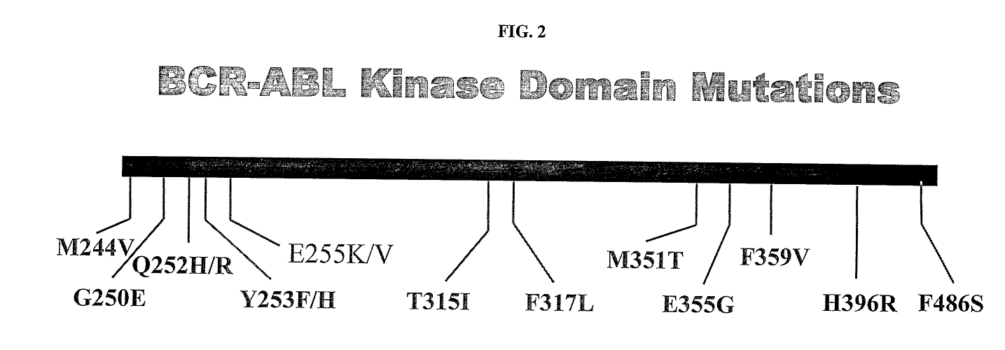

### DETAILED DESCRIPTION OF THE INVENTION

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., 1989, Molecular Cloning: A Laboratory Manual, 2d ed., Cold Spring Harbor Laboratory Press, Cold Spring Harbor, N.Y and Ausubel et al., Current Protocols in Molecular Biology, Wiley Interscience Publishers, (1995). As appropriate, procedures involving the use of commercially available kits and reagents are generally carried out in accordance with manufacturer defined protocols and/or parameters unless otherwise noted.

As used herein, the term “polynucleotide” means a polymeric form of nucleotides of at least about 10 bases or base pairs in length, either ribonucleotides or deoxynucleotides or a modified form of either type of nucleotide, and is meant to include single and double stranded forms of DNA.

As used herein, the term “polypeptide” means a polymer of at least about 6 amino acids. Throughout the specification, standard three letter or single letter designations for amino acids are used.

As used herein, a polynucleotide is said to be “isolated” when it is substantially separated from contaminant polynucleotides that correspond or are complementary to genes other than, for example, the SRC mutant genes or that encode polypeptides other than SRC mutant gene product or fragments thereof. As used herein, a polypeptide is said to be “isolated” when it is substantially separated from contaminant polypeptide that correspond to polypeptides other than the SRC mutant polypeptides or fragments thereof. A skilled artisan can readily employ polynucleotide or polypeptide isolation procedures to obtain said isolated polynucleotides and/or polypeptides.

As used herein, the terms “hybridize”, “hybridizing”, “hybridizes” and the like, used in the context of polynucleotides, are meant to refer to conventional hybridization conditions, preferably such as hybridization in 50% formamide/6×SSC/0.1% SDS/100 μg/ml ssDNA, in which temperatures for hybridization are above 37° C. and temperatures for washing in 0.1×SSC/0.1% SDS are above 55° C., and most preferably to stringent hybridization conditions.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, are known to those of skill in the art and as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) form amide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium. citrate) and 50% formamide at 55° C., followed by a high-stringency wash consisting of 0.1×SSC containing EDTA at 55° C.

“Moderately stringent conditions” may be identified as described by Sambrook et al., 1989, Molecular Cloning: A Laboratory Manual, New York: Cold Spring Harbor Press, and include the use of washing solution and hybridization conditions (e.g., temperature, ionic strength and % SDS) less stringent than those described above. A non-limiting example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

For purposes of shorthand designation of the SRC mutant variants described herein, it is noted that numbers refer to the amino acid residue position along the amino acid sequence of the SRC polypeptide as provided as SEQ ID NO:2. For example, M244 refers to the amino acid methionine at position 244. Amino acid substitutions at a particular position are written as the wild type amino acid, position number, and amino acid substituted therein, in that order. For example, M244V refers to a valine for methionine substitution at position 244. Amino acid identification uses the single-letter alphabet of amino acids, i.e.,

| | | |
|---|---|---|
| Asp | D | Aspartic acid |
| Thr | T | Threonine |
| Ser | S | Serine |
| Glu | E | Glutamic acid |
| Pro | P | Proline |
| Gly | G | Glycine |
| Ala | A | Alanine |
| Cys | C | Cysteine |
| Val | V | Valine |
| Met | M | Methionine |
| Ile | I | Isoleucine |
| Leu | L | Leucine |
| Tyr | Y | Tyrosine |
| Phe | F | Phenylalanine |
| His | H | Histidine |
| Lys | K | Lysine |
| Arg | R | Arginine |
| Trp | W | Tryptophan |
| Gln | Q | Glutamine |
| Asn | N | Asparagine |

In the context of amino acid sequence comparisons, the term “identity” is used to identify and express the percentage of amino acid residues at the same relative positions that are the same. Also in this context, the term “homology” is used to identify and express the percentage of amino acid residues at the same relative positions that are either identical or are similar, using the conserved amino acid criteria of BLAST analysis, as is generally understood in the art. For example, identity and homology values may be generated by WU-BLAST-2 (Altschul et al., Methods in Enzymology, 266: 460-480 (1996): http://blast.wustl/edu/blast/README.html).

“Percent (%) amino acid sequence identity” with respect to the sequences identified herein is defined as the percentage of amino acid residues in a candidate sequence that are identical with the amino acid residues in the SRC sequence, after aligning the sequences and introducing gaps, if necessary, to achieve the maximum percent sequence identity. Alignment for purposes of determining percent amino acid sequence identity can be achieved in various ways that are within the skill in the art and can be determined using appropriate parameters for measuring alignment, including assigning algorithms needed to achieve maximal alignment over the full-length sequences being compared.

Those having skill in the art will know how to determine percent identity between/among sequences using, for example, algorithms such as those based on the CLUSTALW computer program (J. D. Thompson et al., 1994, Nucleic Acids Research, 2(22):4673-4680), or FASTDB, (Brutlag et al., 1990, Comp. App. Biosci., 6:237-245), as known in the art. Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations.

The term “antibody” refers to intact molecules as well as fragments thereof, such as Fab, F(ab′)₂, Fv, which are capable of binding an epitopic or antigenic determinant. Antibodies that bind to SRC or SRC mutant polypeptides can be prepared using intact polypeptides or fragments containing small peptides of interest, which may be prepared recombinantly for use as the immunizing antigen. The polypeptide or oligopeptide used to immunize an animal can be derived from the transition of RNA or synthesized chemically, and can be conjugated to a carrier protein, if desired. Commonly used carriers that are chemically coupled to peptides include, but are not limited to, bovine serum albumin (BSA), keyhole limpet hemocyanin (KLH), and thyroglobulin. The coupled peptide is then used to immunize the animal (e.g., a mouse, a rat, or a rabbit).

The term “antigenic determinant” refers to that portion of a molecule that makes contact with a particular antibody (i.e., an epitope). When a protein or fragment of a protein is used to immunize a host animal, numerous regions of the protein may induce the production of antibodies which bind specifically to a given region or three-dimensional structure on the protein; each of these regions or structures is referred to as an antigenic determinant. An antigenic determinant may compete with the intact antigen (i.e., the immunogen used to elicit the immune response) for binding to an antibody.

The terms “specific binding” or “specifically binding” refer to the interaction between a protein or peptide and a binding molecule, such as an agonist, an antagonist, or an antibody. The interaction is dependent upon the presence of a particular structure (i.e., an antigenic determinant or epitope) of the protein that is recognized by the binding molecule. For purposes of the present invention compounds, for example small molecules, may be considered for their ability to specifically bind to wild type SRC and/or SRC mutants described herein.

The terms “cancer”, “cancerous”, or “malignant” refer to or describe the physiological condition in mammals that is typically characterized by unregulated cell growth. Examples of cancer include but are not limited to, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia (AML), and chronic lymphocytic leukemia (CML).

“Protein tyrosine kinase-associated disorders” are those disorders which result from aberrant tyrosine kinase activity, and/or which are alleviated by the inhibition of one or more of these enzymes. Disorders included in the scope of the present invention may include chronic myeloid leukemia, acute lymphoblastic leukemia, Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia, chronic lymphocytic leukemia, mastocytosis and any symptom associated with mastocytosis. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, and mast cell leukemia. Various cancers are also included within the scope of protein tyrosine kinase-associated disorders including (but not limited to) the following: carcinoma, including that of the bladder, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma.

Protein tyrosine kinase-associated disorders of particular interest herein are those disorders which result, at least in part, from aberrant SRC (WT or mutant) activity and/or which are alleviated by the inhibition of SRC (WT or mutant) referred to herein as “SRC associated disorders” or in the case if cancer “SRC associated cancer.”

“SRC”, “SRC kinase”, and “Mutant SRC kinase” encompasses a SRC kinase with an amino acid sequence that differs from wild type SRC kinase by one or more amino acid substitutions, additions or deletions. For example a substitution of the M244V amino acid with another amino acid would result in a mutant SRC kinase. Encompassed in the present invention are mutant SRC kinases comprising mutations at M244V. Amino acid M244V may be substituted with any of the other available amino acids. In one embodiment M244V is substituted with a non-polar, non-acidic amino acid. Discussed herein are several mutant SRC kinases including those with one or more of the following mutations: M244V, G250E, Q252H, Q252R, Y253F, Y253H, E255K, E255V, T315I, F317L, M351T, E355G, F359V H396R, and F486S. SRC necessarily encompasses BCR/ABL, ABL, SRC including SRC family kinases such as c-Src, SRC/ABL, and other forms including, but not limited to, JAK, FAK, FPS, CSK, SYK, and BTK. Amino acid locations referenced herein are directed to the BCR-ABL Src kinase. Accordingly, the preferred SRC kinase of the present invention is BCR-ABL. However, the invention encompasses the same or similar amino acid changes in relevant amino acid residues of other SRC kinases.

“Mutant SRC kinase associated disorder” is used to describe a protein tyrosine kinase-associated disorder in which the cells involved in said disorder are or become resistant to treatment with a kinase inhibitor used to treat said disorder as a result of a mutation in SRC kinase. As disclosed herein mutations that result in constitutively active SRC kinase are of particular interest. For example, a kinase inhibitor compound may be used to treat a cancerous condition, which compound inhibits the activity of wild type SRC which will inhibit proliferation and/or induce apoptosis of cancerous cells. Over time, a mutation may be introduced into the gene encoding SRC kinase, which may alter the amino acid sequence of SRC kinase and cause the cancer cells to become resistant, or at least partially resistant, to treatment with the compound. Alternatively, a mutation may already be present within the gene encoding SRC kinase, either genetically or as a consequence of an oncogenic event, independent of treatment with a protein tyrosine kinase inhibitor, which may be one factor resulting in these cells propensity to differentiate into a cancerous or proliferative state, and also result in these cells being less sensitive to treatment with a protein tyrosine kinase inhibitor. Such situations are expected to result, either directly or indirectly, in a “mutant SRC kinase associated disorder” and treatment of such condition will require a compound that is at least partially effective against the mutant SRC, preferably against both wild type SRC and the mutant SRC. In the instance where an individual develops at least partial resistance to the kinase inhibitor imatinib, the mutant SRC associated disorder is one that results from an imatinib-resistant SRC mutation, or a protein tyrosine kinase inhibitor resistant SRC mutation.

“imatinib-resistant SRC mutation” “protein tyrosine kinase inhibitor resistant SRC mutation” refers to a specific mutation in the amino acid sequence of SRC that confers upon cells that express said mutation resistance to treatment with imatinib and/or any other protein tyrosine kinase inhibitor. As discussed herein such mutations may include mutations at the M244V position of SRC. Contemplated within the invention is a mutation in the SRC sequence selected from the group consisting of M244V, G250E, Q252H, Q252R, Y253F, Y253H, E255K, E255V, T315I, F317L, M351T, E355G, F359V H396R, and F486S.

The terms “treating”, “treatment” and “therapy” as used herein refer to curative therapy, prophylactic therapy, preventative therapy, and mitigating disease therapy.

The term “mammal” as used herein refers to any mammal classified as a mammal, including humans, cows, horses, dogs and cats. In a preferred embodiment of the invention, the mammal is a human.

The terms “combination”, and “combination of ′N-(2-chloro-6-methylphenyl)-2-[[6-[4-(2-hydroxyethyl)-1-piperazinyl]-2-methyl-4-pyrimidinyl]amino]-5-thiazolecarboxamide” as used herein refers to a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib; combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., COMPOUND II); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide with AMN-107, PD180970, CGP76030, AP23464, SKI 606, AZD0530, AP, and/or a T315I inhibitor including, but not limited to janus kinase inhibitors, AP23848, Jak2 inhibitors, heat shock protein 90 inhibitors, DMAG, VX-680, ON012380, OSU-03012, LBH589, 17-AAG, and combination of LBH589 and 17-AAG; any combinations specifically disclosed in co-owned U.S. Ser. No. 11/402,502, filed Apr. 12, 2006 (hereby incorporated herein by reference); and/or any compounds disclosed and referenced in Deininger et al (Blood, 105(7):2640-2653 (2005); hereby incorporated by reference in its entirety) which include, but are not limited to IFN, pegylated IFN, homoharringtonine, cytabine, hydroxyurea, farnysyl transferase inhibitors, lonafarnib, tipifamib, MEK1 inhibitors, PD98059, RAF-1 inhibitors, BAY43-9006, P13 kinase inhibitors, LY294002, mTOR inhibitors, rapamycin, cyclin-dependent kinase inhibitors, favopiridol; any compounds disclosed and/or referenced in La Rosee et al (Leukemia, 16:1213-1219 (2002), hereby incorporated by reference in its entirety); or pharmaceutically acceptable salts thereof, and optionally in therapeutically effective amounts thereof.

The term “more aggressive dosing regimen”, “increased dosing frequency regimen”, as used herein refers to a dosing regimen that necessarily exceeds the basal and/or prescribed dosing regimen of ′N-(2-chloro-6-methylphenyl)-2-[[6-[4-(2 hydroxyethyl)-1-piperazinyl]-2-methyl-4-pyrimidinyl]amino]-5-thiazolecarboxamide either due to an increased frequency of administration, increased or escalated dose, or the route of administration which may result in an increased bio-available level of ′N-(2-chloro-6-methylphenyl)-2-[[6-[4-(2-hydroxyethyl)-1-piperazinyl]-2-methyl-4-pyrimidinyl]amino]-5-thiazolecarboxamide. Non-limiting examples of such dosing regimens may be found by reference to U.S. Ser. No. 10/395,503, filed Mar. 24, 2003; and Blood (ASH Annual Meeting Abstracts) 2004, Volume 104: Abstract 20, “Hematologic and Cytogenetic Responses in imatinib-Resistant Accelerated and Blast Phase Chronic Myeloid Leukemia (CML) Patients Treated with the Dual SRC/ABL Kinase Inhibitor ′N-(2-chloro-6-methylphenyl)-2-[[6-[4-(2-hydroxyethyl)-1-piperazinyl]-2-methyl-4-pyrimidinyl]amino]-5-thiazolecarboxamide: Results from a Phase I Dose Escalation Study.”, by Moshe Talpaz, et al; and/or dosing regimens outlined in Deininger et al (Blood, 105(7):2640-2653 (2005); hereby incorporated by reference in its entirety); which are hereby incorporated herein by reference.

Additional definitions are provided throughout the specification.

The invention provides a method of treating cancers, including both primary and metastatic cancers, including solid tumors such as those of the breast, colon, and prostate, as well as lymphomas and leukemias (including CML, AML and ALL), cancers of endothelial tissues, and including cancers which are resistant to other therapies, including other therapies involving administration of kinase inhibitors such as imatinib. Specifically, and without limitation, the invention provides the use of a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib; a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., COMPOUND II); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; any other combination disclosed herein; an increased dosing frequency regimen of ′N-(2-chloro-6-methylphenyl)-2-[[6-[4-(2-hydroxyethyl)-1-piperazinyl]-2-methyl-4-pyrimidinyl]amino]-5-thiazolecarboxamide; and any other combination or dosing regimen comprising ′N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein, for treating disorders, for example cancers, which are resistant to other therapies involving administration of kinase inhibitors such as imatinib.

The invention provides that a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib; a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., COMPOUND II); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; any other combination disclosed herein; an increased dosing frequency regimen of ′N-(2-chloro-6-methylphenyl)-2-[[6-[4-(2-hydroxyethyl)-1-piperazinyl]-2-methyl-4-pyrimidinyl]amino]-5-thiazolecarboxamide; and any other combination or dosing regimen comprising ′N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein, will be useful against cancers which are resistant or at least partially resistant to one or more other anticancer agents, including among others cancers which are resistant in whole or part to other anticancer agents, specifically including imatinib and other kinase inhibitors, and specifically including cancers involving one or more mutations in SRC kinase.

The invention also contemplates a method for identifying an individual for treatment comprising screening cells from an individual to identify a mutant SRC kinase expressed in said cells, and if a mutant SRC mutant kinase is present, administering a therapeutically effective amount of an inhibitor of said mutant SRC kinase, or an increased therapeutically effective amount of an inhibitor of said mutant SRC kinase. According to the present invention, and without limitation, the identification of at least one mutation at the following positions of the SRC kinase of SEQ ID NO:2, would indicate an individual selected for treatment: M244V, G250E, Q252H, Q252R, Y253F, Y253H, E255K, E255V, T315I, F317L, M351T, E355G, F359V H396R, and F486S.

Methods of identifying the amino acid and nucleic acid of a mutant SRC kinase are known in the art. Standard molecular biology techniques are contemplated for precisely determining a SRC mutation in the cells of a given individual.

Antibodies that immunospecifically bind to a mutant SRC kinase may be used in identifying one or more of the SRC mutants described herein. Contemplated herein are antibodies that specifically bind to a mutant SRC kinase and that do not bind (or bind weakly) to wild type SRC protein or polypeptides. Anti-mutant SRC kinase antibodies include monoclonal and polyclonal antibodies as well as fragments containing the antigen binding domain and/or one or more complementarity determining regions of these antibodies.

For some applications, it may be desirable to generate antibodies which specifically react with a particular mutant SRC kinase protein and/or an epitope within a particular structural domain. For example, preferred antibodies useful for diagnostic purposes are those which react with an epitope in a mutated region of the SRC protein as expressed in cancer cells. For example, antibodies that bind specifically to a M244V, G250E, Q252H, Q252R, Y253F, Y253H, E255K, E255V, T315I, F317L, M351T, E355G, F359V H396R, or F486S mutant SRC kinase. Such antibodies may be generated by using the mutant SRC kinase protein described herein, or using peptides derived from various domains thereof, as an immunogen.

Mutant SRC kinase antibodies of the invention may be particularly useful in cancer (e.g., chronic myeloid leukemia, acute lymphoblastic leukemia, Philadelphia chromosome positive acute lymphoblastic leukemia (Ph+ ALL, GIST) therapeutic strategies, diagnostic and prognostic assays, and imaging methodologies. Similarly, such antibodies may be useful in the diagnosis, and/or prognosis of other cancers, to the extent such mutant SRC kinase is also expressed or overexpressed in other types of cancer. The invention provides various immunological assays useful for the detection and quantification of mutant SRC kinase proteins and polypeptides. Such assays generally comprise one or more mutant SRC kinase antibodies capable of recognizing and binding a mutant SRC kinase protein, as appropriate, and may be performed within various immunological assay formats well known in the art, including but not limited to various types of radioimmunoassays, enzyme-linked immunosorbent assays (ELISA), enzyme-linked immunofluorescent assays (ELIFA), and the like. In addition, immunological imaging methods capable of detecting cancer cells are also provided by the invention, including but not limited to imaging methods using labeled mutant SRC kinase antibodies. Such assays may be used clinically in the detection, monitoring, and prognosis of cancers.

Another aspect of the present invention relates to methods for detecting mutant SRC kinase polynucleotides and mutant SRC kinase proteins, as well as methods for identifying a cell that expresses mutant SRC kinase. The expression profile of mutant SRC kinases makes them diagnostic markers for disease states. The status of mutant SRC kinase gene products in patient samples may be analyzed by a variety protocols that are well known in the art including the following non-limiting types of assays: PCR-free genotyping methods, Single-step homogeneous methods, Homogeneous detection with fluorescence polarization, Pyrosequencing, “Tag” based DNA chip system, Bead-based methods, fluorescent dye chemistry, Mass spectrometry based genotyping assays, TaqMan genotype assays, Invader genotype assays, microfluidic genotype assays, immunohistochemical analysis, the variety of Northern blotting techniques including in situ hybridization, RT-PCR analysis (for example on laser capture micro-dissected samples), western blot analysis, tissue array analysis, and any other methods known in the art or described elsewhere herein.

Specifically encompassed by the present invention are the following, non-limiting genotyping methods: Landegren, U., Nilsson, M. & Kwok, P. Genome Res 8, 769-776 (1998); Kwok, P., Pharmacogenomics 1, 95-100 (2000); Gut, I., Hum Mutat 17, 475-492 (2001); Whitcombe, D., Newton, C. & Little, S., Curr Opin Biotechnol 9, 602-608 (1998); Tillib, S. & Mirzabekov, A., Curr Opin Biotechnol 12, 53-58 (2001); Winzeler, E. et al., Science 281, 1194-1197 (1998); Lyamichev, V. et al., Nat Biotechnol 17, 292-296 (1999); Hall, J. et al., Proc Natl Acad Sci USA 97, 8272-8277 (2000); Mein, C. et al., Genome Res 10, 333-343 (2000); Ohnishi, Y. et al., J Hum Genet. 46, 471-477 (2001); Nilsson, M. et al., Science 265, 2085-2088 (1994); Baner, J., Nilsson, M., Mendel-Hartvig, M. & Landegren, U., Nucleic Acids Res 26, 5073-5078 (1998); Baner, J. et al., Curr Opin Biotechnol 12, 11-15 (2001); Hatch, A., Sano, T., Misasi, J. & Smith, C., Genet Anal 15, 35-40 (1999); Lizardi, P. et al., Nat Genet. 19, 225-232 (1998); Zhong, X., Lizardi, P., Huang, X., Bray-Ward, P. & Ward, D., Proc Natl Acad Sci USA 98, 3940-3945 (2001); Faruqi, F. et al. BMC Genomics 2, 4 (2001); Livak, K., Gnet Anal 14, 143-149 (1999); Marras, S., Kramer, F. & Tyagi, S., Genet Anal 14, 151-156 (1999); Ranade, K. et al., Genome Res 11, 1262-1268 (2001); Myakishev, M., Khripin, Y., Hu, S. & Hamer, D., Genome Re 11, 163-169 (2001); Beaudet, L., Bedard, J., Breton, B., Mercuri, R. & Budarf, M., Genome Res 11, 600-608 (2001); Chen, X., Levine, L. & PY, K., Genome Res 9, 492-498 (1999); Gibson, N. et al., Clin Chem 43, 1336-1341 (1997); Latif, S., Bauer-Sardina, I., Ranade, K., Livak, K. & PY, K., Genome Res 11, 436-440 (2001); Hsu, T., Law, S., Duan, S., Neri, B. & Kwok, P., Clin Chem 47, 1373-1377 (2001); Alderboni, A., Kristofferson, A. & Hammerling, U., Genonie Res 10, 1249-1258 (2000); Ronaghi, M., Uhlen, M. & Nyren, P., Science 281, 363, 365 (1998); Ronaghi, M., Genome Res 11, 3-11 (2001); Pease, A. et al., Proc Natl Acad Sci USA 91, 5022-5026 (1994); Southern, E., Maskos, U. & Elder, J., Genomics 13, 1008-1017 (1993); Wang, D. et al., Science 280, 1077-1082 (1998); Brown, P. & Botstein, D., Nat Genet. 21, 33-37 (1999); Cargill, M. et al. Nat Genet. 22, 231-238 (1999); Dong, S. et al., Genome Res 11, 1418-1424 (2001); Halushka, M. et al., Nat Genet. 22, 239-247 (1999); Hacia, J., Nat Genet. 21, 42-47 (1999); Lipshutz, R., Fodor, S., Gingeras, T. & Lockhart, D., Nat Genet. 21, 20-24 (1999); Sapolsky, R. et al., Genet Anal 14, 187-192 (1999); Tsuchihashi, Z. & Brown, P., J Virol 68, 5863 (1994); Herschlag, D., J Biol Chem 270, 20871-20874 (1995); Head, S. et al., Nucleic Acids Res 25, 5065-5071 (1997); Nikiforov, T. et al., Nucleic Acids Res 22, 4167-4175 (1994); Syvanen, A. et al., Genomics 12, 590-595 (1992); Shurnaker, J., Metspalu, A. & Caskey, C., Hum Mutat 7, 346-354 (1996); Lindroos, K., Liljedahl, U., Raitio, M. & Syvanen, A., Nucleic Acids Res 29, E69-9 (2001); Lindblad-Toh, K. et al., Nat Genet. 24, 381-386 (2000); Pastinen, T. et al., Genome Res 10, 1031-1042 (2000); Fali, J. et al., Genome Res 10, 853-860 (2000); Hirschhorn, J. et al., Proc Natl Acad Sci USA 97, 12164-12169 (2000); Bouchie, A., Nat Biotechnol 19, 704 (2001); Hensel, M. et al., Science 269, 400-403 (1995); Shoemaker, D., Lashkari, D., Morris, D., Mittmann, M. & Davis, R. Nat Genet. 14, 450-456 (1996); Gerry, N. et al., J Mol Biol 292, 251-262 (1999); Ladner, D. et al., Lab Invest 81, 1079-1086 (2001); lannone, M. et al. Cytometry 39, 131-140 (2000); Fulton, R., McDade, R., Smith, P., Kienker, L. & Kettman, J. J., Clin Chem 43, 1749-1756 (1997); Armstrong, B., Stewart, M. & Mazumder, A., Cytometry 40, 102-108 (2000); Cai, H. et al., Genomics 69, 395 (2000); Chen, J. et al., Genome Res 10, 549-557 (2000); Ye, F. et al. Hum Mutat 17, 305-316 (2001); Michael, K., Taylor, L., Schultz, S. & Walt, D., Anal Chem 70, 1242-1248 (1998); Steemers, F., Ferguson, J. & Walt, D., Nat Biotechnol 18, 91-94 (2000); Chan, W. & Nie, S., Science 281, 2016-2018 (1998); Han, M., Gao, X., Su, J. & Nie, S., Nat Biotechnol 19, 631-635 (2001); Griffin, T. & Smith, L., Trends Biotechnol 18, 77-84 (2000); Jackson, P., Scholl, P. & Groopman, J., Mol Med Today 6, 271-276 (2000); Haff, L. & Smirnov, I., Genome Res 7, 378-388 (1997); Ross, P., Hall, L., Smirnov, I. & Haff, L., Nat Biotechnol 16, 1347-1351 (1998); Bray, M., Boerwinkle, E. & Doris, P. Hum Mutat 17, 296-304 (2001); Sauer, S. et al., Nucleic Acids Res 28, E13 (2000); Sauer, S. et al., Nucleic Acids Res 28, E100 (2000); Sun, X., Ding, H., Hung, K. & Guo, B., Nucleic Acids Res 28, E68 (2000); Tang, K. et al., Proc Natl Acad Sci USA 91, 10016-10020 (1999); Li, J. et al., Electrophoresis 20, 1258-1265 (1999); Little, D., Braun, A., O'Donnell, M. & Koster, H., Nat Med 3, 1413-1416 (1997); Little, D. et al. Anal Chem 69, 4540-4546 (1997); Griffin, T., Tang, W. & Smith, L., Nat Biotechnol 15, 1368-1372 (1997); Ross, P., Lee, K. & Belgrader, P., Anal Chem 69, 4197-4202 (1997); Jiang-Baucom, P., Girard, J., Butler, J. & Belgrader, P., Anal Chem 69, 4894-4898 (1997); Griffin, T., Hall, J., Prudent, J. & Smith, L., Proc Natl Acad Sci USA 96, 6301-6306 (1999); Kokoris, M. et al., Mol Diagn 5, 329-340 (2000); Jurinke, C., van den Boom, D., Cantor, C. & Koster, H. (2001); and/or Taranenko, N. et al., Genet Anal 13, 87-94 (1996).

The following additional genotyping methods are also encompassed by the present invention: the methods described and/or claimed in U.S. Pat. No. 6,458,540; and the methods described and/or claimed in U.S. Pat. No. 6,440,707.

Probes and primers can be designed so as to be specific to such mutation analysis and are derived from the wild type SRC sequence, segments and complementary sequences thereof.

Additionally, the invention provides assays for the detection of mutant SRC kinase polynucleotides in a biological sample, such as cell preparations, and the like. A number of methods for amplifying and/or detecting the presence of mutant SRC kinase polynucleotides are well known in the art and may be employed in the practice of this aspect of the invention.

In one embodiment, a method for detecting a mutant SRC kinase mRNA in a biological sample comprises producing cDNA from the sample by reverse transcription using at least one primer; amplifying the cDNA so produced using mutant SRC kinase polynucleotides as sense and antisense primers to amplify mutant SRC kinase cDNAs therein; and detecting the presence of the amplified mutant SRC kinase cDNA. Any number of appropriate sense and antisense probe combinations may be designed from the nucleotide sequences provided for a mutant SRC kinase and used for this purpose.

The invention also provides assays for detecting the presence of a mutant SRC kinase protein in a biological sample. Methods for detecting a mutant SRC kinase protein are also well known and include, for example, immunoprecipitation, immunohistochemical analysis, Western Blot analysis, molecular binding assays, ELISA, ELIFA and the like. For example, in one embodiment, a method of detecting the presence of a mutant SRC kinase protein in a biological sample comprises first contacting the sample with a SRC antibody, a mutant SRC kinase-reactive fragment thereof, or a recombinant protein containing an antigen binding region of a mutant SRC kinase antibody; and then detecting the binding of mutant SRC kinase protein in the sample thereto.

Methods for identifying a cell that expresses mutant SRC kinase are also provided. In one embodiment, an assay for identifying a cell that expresses a mutant SRC kinase gene comprises detecting the presence of mutant SRC kinase mRNA in the cell. Methods for the detection of particular mRNAs in cells are well known and include, for example, hybridization assays using complementary DNA probes (such as in situ hybridization using labeled mutant SRC kinase riboprobes, Northern blot and related techniques) and various nucleic acid amplification assays (such as RT-PCR using complementary primers specific for a mutant SRC kinase, and other amplification type detection methods, such as, for example, branched DNA, SISBA, TMA and the like).

The invention encompasses treatment methods based upon the demonstration that patients harboring different SRC kinase mutations, particularly BCR/ABL mutations, have varying degrees of resistance and/or sensitivity to imatinib and ′N-(2-chloro-6-methylphenyl)-2-[[6-[4-(2-hydroxyethyl)-1-piperazinyl]-2-methyl-4-pyrimidinyl]amino]-5-thiazolecarboxamide, respectively. Thus the methods of the present invention can be used in determining whether or not to treat an individual with a combination of tyrosine kinase inhibitors, such as ′N-(2-chloro-6-methylphenyl)-2-[[6-[4-(2-hydroxyethyl)-1-piperazinyl]-2-methyl-4-pyrimidinyl]amino]-5-thiazolecarboxamide and a BCR-ABL inhibitor (e.g., such as imatinib or AMN107); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., COMPOUND II); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; any other combination disclosed herein; an increased dosing frequency regimen of ′N-(2-chloro-6-methylphenyl)-2-[[6-[4-(2-hydroxyethyl)-1-piperazinyl]-2-methyl-4-pyrimidinyl]amino]-5-thiazolecarboxamide; and any other combination or dosing regimen comprising ′N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein. One embodiment of the invention is a method of identifying a mutation in a SRC polynucleotide in a mammalian cell, wherein the mutation in a SRC polynucleotide is associated with resistance to inhibition of SRC kinase activity by imatinib, the method comprising determining the sequence of at least one SRC kinase polynucleotide expressed by the mammalian cell and comparing the sequence of the SRC kinase polynucleotide to the wild type SRC kinase polynucleotide sequence. As described herein the polynucleotide identified may encode a polynucleotide having at least one amino acid difference from the wild type SRC kinase amino acid sequence.

Treatment regimens may be established based upon the presence of one or more mutant SRC kinases disclosed herein. For example, the invention encompasses screening cells from an individual who may suffer from, or is suffering from, a disorder that is commonly treated with a kinase inhibitor. Such a disorder may include myeloid leukemia or disorders associated therewith, or cancers described herein. The cells of an individual are screened, using methods known in the art, for identification of a mutation in a SRC kinase. Mutations of interest are those that result in SRC kinase being constitutively activated. Specific mutations include M244V (wherein the methionine at position 244 is replaced with a valine), G250E (wherein the glycine at position 250 is replaced with a glutamic acid), Q252H (wherein the glutamine at position 252 is replaced with a histidine), Q252R (wherein the glutamine at position 252 is replaced with an arginine), Y253F (wherein the tyrosine at position 252 is replaced with a phenylalanine), Y253H (wherein the tyrosine at position 252 is replaced with a histidine), E255K (wherein the glutamic acid at position 255 is replaced with a lysine), E255V (wherein the glutamic acid at position 255 is replaced with a valine), T315I (wherein the threonine at position 315 is replaced with a isoleucine), F317L (wherein the phenylalanine at position 317 is replaced with a leucine), M351T (wherein the methionine at position 315 is replaced with a threonine), E355G (wherein the glutamic acid at position 355 is replaced with a glycine), F359V (wherein the phenylalanine at position 359 is replaced with a valine), H396R (wherein the histidine at position 396 is replaced with a arginine), and F486S (wherein the phenylalanine at position 486 is replaced with a serine) of SEQ ID NO:2.

If an activating SRC kinase mutation is found in the cells from said individual, treatment regimens can be developed appropriately. For example, an identified mutation may indicate that said cells are or will become resistant to commonly used kinase inhibitors. Such an inhibitor includes the kinase inhibitor imatinib. If the cells in an individual are or are expected to become resistant to treatment with a kinase inhibitor such as imatinib, then, as disclosed herein, treatment should include the use of a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib; a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., COMPOUND II); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; any other combination disclosed herein; an increased dosing frequency regimen of ′N-(2-chloro-6-methylphenyl)-2-[[6-[4-(2-hydroxyethyl)-1-piperazinyl]-2-methyl-4-pyrimidinyl]amino]-5-thiazolecarboxamide; and any other combination or dosing regimen comprising ′N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein.

Additionally, dosage regimens may be established based upon the presence of a specific amino acid mutation in a SRC kinase. As disclosed herein, a specific amino acid mutation in SRC kinase may make said mutant SRC kinase more sensitive to treatment with a kinase inhibitor than another amino acid substitution. As described, cells exhibiting the Q252R mutant SRC kinase are more sensitive to treatment with ′N-(2-chloro-6-methylphenyl)-2-[[6-[4-(2-hydroxyethyl)-1-piperazinyl]-2-methyl-4-pyrimidinyl]amino]-5-thiazolecarboxamide than cells that exhibit the F317L mutant SRC kinase. This discovery is useful in determining the dosage regimen for an individual whose cells exhibit such a mutation. For example, a less aggressive dosage regimen of inhibitor ′N-(2-chloro-6-methylphenyl)-2-[[6-[4-(2-hydroxyethyl)-1-piperazinyl]-2-methyl-4-pyrimidinyl]amino]-5-thiazolecarboxamide may be employed if cells exhibit a Q252R mutant SRC kinase; alternatively a more aggressive dosage regimen of inhibitor ′N-(2-chloro-6-methylphenyl)-2-[[6-[4-(2-hydroxyethyl)-1-piperazinyl]-2-methyl-4-pyrimidinyl]amino]-5-thiazolecarboxamide may be employed alone or in combination with other agents disclosed herein, if cells exhibit a F317L mutant kinase. In one embodiment, an increased or decreased level of dasatinib would be about 10, 20, 30, 40, 50, 60, 70, 80, 90, or 95% more than the typical dasatinib dose for a particular indication or for individual, or about 1.5×, 2×, 2.5×, 3×, 3.5×, 4×, 4.5×, or 5× more dasatinib than the typical dasatinib dose for a particular indication or for individual.

A therapeutically effective amount of Compound I can be orally administered as an acid salt of Compound I. The actual dosage employed may be varied depending upon the requirements of the patient and the severity of the condition being treated. Determination of the proper dosage for a particular situation is within the skill of the art. The effective amount of Compound I (and Compound I salt) may be determined by one of ordinary skill in the art, and includes exemplary dosage amounts for an adult human of from about 0.05 to about 100 mg/kg of body weight of Compound I per day, which may be administered in a single dose or in the form of individual divided doses, such as from 1, 2, 3, or 4 times per day. In one embodiment, Compound I is administered 2 times per day at 70 mg. Alternatively, it may be dosed at 50, 70, 90, 100, 110, or 120 BID, or 100, 140, or 180 once daily. It will be understood that the specific dose level and frequency of dosing for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, and the like, subject to protein tyrosine kinase-associated disorders. The same also applies to Compound II or any combination of Compound I and II, or any combination disclosed herein.

In the method disclosed above, the mammalian cell may be a human cancer cell. The human cancer cell may be one obtained from an individual treated with imatinib. Optionally the amino acid substitution in the SRC polypeptide expressed in said human cancer cell confers resistance to inhibition of tyrosine kinase activity by imatinib.

A method of determining the responsiveness of an individual suffering from a protein tyrosine kinase-associated disorder to a combination of kinase inhibitors, such as ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib, is also disclosed herein. For example, an individual may be determined to be a positive responder (or cells from said individual would be expected to have a degree of sensitivity) to a certain kinase inhibitor based upon the presence of a mutant SRC kinase. As disclosed herein, cells that exhibit certain mutations at amino acid positions 250, 252, 253, 255, 315, 317, 351, 355, 359, 396 and 486 of SRC kinase, particularly of SEQ ID NO:2, develop resistance to imatinib. Therefore, individuals suffering from a protein tyrosine kinase-associated disorder whose cells exhibit such a mutation are or would be expected to be negative responders to treatment with imatinib. An individual whose cells exhibit a mutant BCR/ABL kinase as disclosed herein may be identified as a positive responder to a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib, or other combinations disclosed herein, in spite of a negative response to imatinib. Treatment regimens are then established for such individuals accordingly.

Treatment regimens based upon the presence of a mutant SRC kinase are also provided herein. A treatment regimen is a course of therapy administered to an individual suffering from a protein kinase associated disorder that may include treatment with one or more kinase inhibitors, as well as other therapies such as radiation and/or other agents. When more than one therapy is administered, the therapies may be administered concurrently or consecutively (for example, more than one kinase inhibitor may be administered together or at different times, on a different schedule). Administration of more than one therapy may be at different times (i.e., consecutively) and still be part of the same treatment regimen. As disclosed herein, for example, cells from an individual suffering from a protein kinase associated disorder may be found to develop resistance to imatinib. Based upon the present discovery that such cells may be sensitive to a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib a treatment regimen can be established that includes treatment with the combination either as a monotherapy, or in combination with another kinase inhibitor, or in combination with another agent as disclosed herein. Additionally, the combination can be administered with radiation or other known treatments.

Therefore the present invention includes a method for establishing a treatment regimen for an individual suffering from a protein tyrosine kinase associated disorder comprising: (a) providing a sample of cells from said individual; (b) screening said sample of cells for the presence of at least one mutation in a SRC kinase sequence; and, if at least one mutation in a SRC kinase sequence is present in said sample of cells, (c) administering to said individual as part of a treatment regimen a pharmaceutical composition comprising ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib; a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., COMPOUND II); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; any other combination disclosed herein; an increased dosing frequency regimen of ′N-(2-chloro-6-methylphenyl)-2-[[6-[4-(2-hydroxyethyl)-1-piperazinyl]-2-methyl-4-pyrimidinyl]amino]-5-thiazolecarboxamide; and any other combination or dosing regimen comprising ′N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein. When screening the cells from an individual suffering from a protein kinase associated disorder one may look for a mutation in the nucleic acid sequence encoding SRC kinase, or for a resulting mutation in the amino acid sequence of SRC kinase.

Wherever the term “′N-(2-chloro-6-methylphenyl)-2-[[6-[4-(2-hydroxyethyl)-1-piperazinyl]-2-methyl-4-pyrimidinyl]amino]-5-thiazolecarboxamide” (e.g., Compound I) is used herein, it is understood (unless otherwise indicated) that the compound ′N-(2-chloro-6-methylphenyl)-2-[[6-[4-(2-hydroxyethyl)-1-piperazinyl]-2-methyl-4-pyrimidinyl]amino]-5-thiazolecarboxamide having the following structure (I):

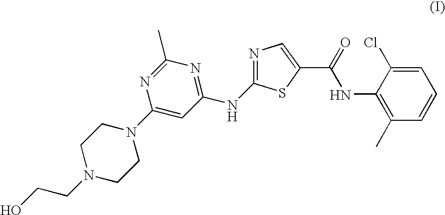

is intended, as well as all pharmaceutically acceptable salts thereof. Compound (I) is also referred to as N-(2-chloro-6-methylphenyl)-2-((6-(4-(2-hydroxyethyl)-1-piperazinyl)-2-methyl-4-pyrimidinyl)amino)-1,3-thiazole-5-carboxamide in accordance with IUPAC nomenclature. Use of the term encompasses (unless otherwise indicated) solvates (including hydrates) and polymorphic forms of the compound (I) or its salts (such as the monohydrate form of (I) described in U.S. Ser. No. 11/051,208, filed Feb. 4, 2005, incorporated herein by reference). Pharmaceutical compositions of ′N-(2-chloro-6-methylphenyl)-2-[[6-[4-(2-hydroxyethyl)-1-piperazinyl]-2-methyl-4-pyrimidinyl]amino]-5-thiazolecarboxamide include all pharmaceutically acceptable compositions comprising ′N-(2-chloro-6-methylphenyl)-2-[[6-[4-(2-hydroxyethyl)-1-piperazinyl]-2-methyl-4-pyrimidinyl]amino]-5-thiazolecarboxamide and one or more diluents, vehicles and/or excipients, such as those compositions described in U.S. Ser. No. 11/402,502, filed Apr. 12, 2006, incorporated herein by reference.

Wherein the term “a farnysyl transferase inhibitor” (e.g., Compound II) herein, it is understood (unless otherwise indicated) that the compound have formula (II), (R)-2,3,4,5-tetrahydro-1(1H-imidazol-4-ylmethyl)-3-(phenylmethyl)-4-(2-thienylsulfonyl)-1H-1H,4-benzodiazepine-7-carbonitrile, hydrochloride salt, is an anti-cancer agent. The compound of formula (II) is a cytotoxic FT inhibitor which is known to kill non-proliferating cancer cells preferentially. The compound of formula (II) may further be useful in killing stem cells.

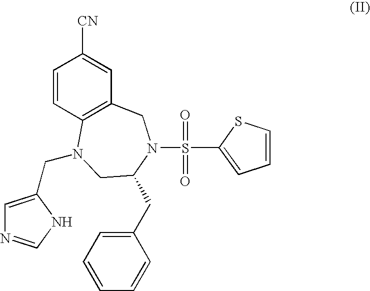

The compound of formula (II), its preparation, and uses thereof are described in U.S. Pat. No. 6,011,029, which is herein incorporated by reference. The uses of the compound of formula (II) are also described in WO2004/015130, published Feb. 19, 2004, which is herein incorporated by reference.

In practicing the many aspects of the invention herein, biological samples may be selected from many sources such as tissue biopsy (including cell sample or cells cultured therefrom; biopsy of bone marrow or solid tissue, for example cells from a solid tumor), blood, blood cells (red blood cells or white blood cells), serum, plasma, lymph, ascetic fluid, cystic fluid, urine, sputum, stool, saliva, bronchial aspirate, CSF or hair. Cells from a sample may be used, or a lysate of a cell sample may be used.

Analysis of the expression of a mutant SRC kinase described herein may also be useful as a tool for identifying and evaluating agents that modulate expression of a nucleic acid sequence encoding mutant SRC kinase, for example, an antisense or sRNAi polynucleotide that would block expression of mutant SRC kinase. Identification of a molecule of biologic agent that could inhibit mutant SRC kinase activity is of therapeutic value. An agent that specifically binds to a nucleic acid sequence encoding SRC wherein a mutation selected from the group consisting of M244V, G250E, Q252H, Q252R, Y253F, Y253H, E255K, E255V, T315I, F317L, M351T, E355G, F359V H396R, and F486S is of therapeutic value.

Pharmaceutical compositions for use in the present invention include compositions comprising a combination of inhibitors of a mutant SRC kinase in an effective amount to achieve the intended purpose. The determination of an effective dose of a pharmaceutical composition of the invention is well within the capability of those skilled in the art. A therapeutically effective dose refers to that amount of active ingredient which ameliorates the symptoms or condition. Therapeutic efficacy and toxicity may be determined by standard pharmaceutical procedures in cell cultures or experimental animals, for example the ED50 (the dose therapeutically effective in 50% of the population) and LD50 (the dose lethal to 50% of the population).

Dosage regimens involving ′N-(2-chloro-6-methylphenyl)-2-[[6-[4-(2-hydroxyethyl)-1-piperazinyl]-2-methyl-4-pyrimidinyl]amino]-5-thiazolecarboxamide useful in practicing the present invention are described in U.S. Ser. No. 10/395,503, filed Mar. 24, 2003; and Blood (ASH Annual Meeting Abstracts) 2004, Volume 104: Abstract 20, “Hematologic and Cytogenetic Responses in imatinib-Resistant Accelerated and Blast Phase Chronic Myeloid Leukemia (CML) Patients Treated with the Dual SRC/ABL Kinase Inhibitor ′N-(2-chloro-6-methylphenyl)-2-[[6-[4-(2-hydroxyethyl)-1-piperazinyl]-2-methyl-4-pyrimidinyl]amino]-5-thiazolecarboxamide: Results from a Phase I Dose Escalation Study.”, by Moshe Talpaz, et al; which are hereby incorporated herein by reference.

A “therapeutically effective amount” of an inhibitor of a mutant SRC kinase may be a function of the mutation present. For example Shah et al disclose cell lines with certain mutations in SRC kinase are more sensitive to ′N-(2-chloro-6-methylphenyl)-2-[[6-[4-(2-hydroxyethyl)-1-piperazinyl]-2-methyl-4-pyrimidinyl]amino]-5-thiazolecarboxamide than cell lines with different SRC kinase mutations. As disclosed therein, cells comprising a F317L mutation in SRC kinase requires three to five-fold higher concentration of ′N-(2-chloro-6-methylphenyl)-2-[[6-[4-(2-hydroxyethyl)-1-piperazinyl]-2-methyl-4-pyrimidinyl]amino]-5-thiazolecarboxamide than cell lines expressing a Q252R mutation. One skilled in the art will appreciate the difference in sensitivity of the mutant SRC kinase cells and determine a therapeutically effective dose accordingly.

Examples of predicted therapeutically effective doses of ′N-(2-chloro-6-methylphenyl)-2-[[6-[4-(2-hydroxyethyl)-1-piperazinyl]-2-methyl-4-pyrimidinyl]amino]-5-thiazolecarboxamide that may be warranted based upon the relative sensitivity of SRC kinase mutants to ′N-(2-chloro-6-methylphenyl)-2-[[6-[4-(2-hydroxyethyl)-1-piperazinyl]-2-methyl-4-pyrimidinyl]amino]-5-thiazolecarboxamide compared to wild-type SRC kinase in various in vitro biochemical assays including cellular proliferation, BCR-Abl tyrosine phosphorylation, peptide substrate phosphorylation, and/or autophosphorylation assays. Approximate therapeutically effective doses of ′N-(2-chloro-6-methylphenyl)-2-[[6-[4-(2-hydroxyethyl)-1-piperazinyl]-2-methyl-4-pyrimidinyl]amino]-5-thiazolecarboxamide may be calculated based upon multiplying the typical dose with the fold change in sensitivity in anyone or more of these assays for each SRC kinase mutant. O'Hare et al. (Cancer Research, 65(11):4500-5 (2005), which is hereby incorporated by reference in its entirety) performed analysis of the relative sensitivity of ′N-(2-chloro-6-methylphenyl)-2-[[6-[4-(2-hydroxyethyl)-1-piperazinyl]-2-methyl-4-pyrimidinyl]amino]-5-thiazolecarboxamide with several clinically relevant SRC Kinase mutants. For example, the E255V mutant had a fold change of “1” in the GST-Abl kinase assay, whereas this same mutant had a fold change of “14” in the cellular proliferation assay. Thus, a therapeutically relevant dose of ′N-(2-chloro-6-methylphenyl)-2-[[6-[4-(2-hydroxyethyl)-1-piperazinyl]-2-methyl-4-pyrimidinyl]amino]-5-thiazolecarboxamide for patients harboring this mutation could range anywhere from 1 to 14 fold higher than the typical dose. Accordingly, therapeutically relevant doses of ′N-(2-chloro-6-methylphenyl)-2-[[6-[4-(2-hydroxyethyl)-1-piperazinyl]-2-methyl-4-pyrimidinyl]amino]-5-thiazolecarboxamide for any of the SRC kinase mutants may be 1, 2, 3, 4, 5, 6, 7, 8, 9, 10, 11, 12, 13, 14, 15, 16, 17, 18, 19, 20, 25, 30, 35, 40, 50, 60, 70, 80, 90, 100, 125, 150, 175, 200, 225, 250, or 300 folder higher than the prescribed dose. Alternatively, therapeutically relevant doses of ′N-(2-chloro-6-methylphenyl)-2-[[6-[4-(2-hydroxyethyl)-1-piperazinyl]-2-methyl-4-pyrimidinyl]amino]-5-thiazolecarboxamide may be 0.9, 0.8, 0.7, 0.6, 0.5, 0.4, 0.3, 0.2, 0.1, 0.09, 0.08, 0.07, 0.06, 0.05, 0.04, 0.03, 0.02, or 0.01 of the prescribed dose.

According to O'hare et al., the M244V mutant had a fold change of “1.3” in the GST-Abl kinase assay, a fold change of “1.1” in the autophosphorylation assay, and a fold change of “2” in the cellular proliferation assay; the G250E mutant had a fold change of “0.5” in the GST-Abl kinase assay, a fold change of “3” in the autophosphorylation assay, and a fold change of “2” in the cellular proliferation assay; the Q252H mutant had a fold change of “4” in the cellular proliferation assay; the Y253F mutant had a fold change of “0.6” in the GST-Abl kinase assay, a fold change of “4” in the autophosphorylation assay, and a fold change of “4” in the cellular proliferation assay; the Y253H mutant had a fold change of “3” in the GST-Abl kinase assay, a fold change of “2” in the autophosphorylation assay, and a fold change of “2” in the cellular proliferation assay; the E255K mutant had a fold change of “0.3” in the GST-Abl kinase assay, a fold change of “2” in the autophosphorylation assay, and a fold change of “7” in the cellular proliferation assay; the F317L mutant had a fold change of “1.5” in the GST-Abl kinase assay, a fold change of “1.4” in the autophosphorylation assay, and a fold change of “9” in the cellular proliferation assay; the M351T mutant had a fold change of “0.2” in the GST-Abl kinase assay, a fold change of “2” in the autophosphorylation assay, and a fold change of “1.4” in the cellular proliferation assay; the F359V mutant had a fold change of “0.8” in the GST-Abl kinase assay, a fold change of “2” in the autophosphorylation assay, and a fold change of “3” in the cellular proliferation assay; the H396R mutant had a fold change of “1.3” in the GST-Abl kinase assay, a fold change of “3” in the autophosphorylation assay, and a fold change of “2” in the cellular proliferation assay.

For patients harboring the T315I mutation, administration of higher doses of ′N-(2-chloro-6-methylphenyl)-2-[[6-[4-(2-hydroxyethyl)-1-piperazinyl]-2-methyl-4-pyrimidinyl]amino]-5-thiazolecarboxamide, or combinations of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib; a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., COMPOUND II); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; any other combination discloses herein; an increased dosing frequency regimen of ′N-(2-chloro-6-methylphenyl)-2-[[6-[4-(2-hydroxyethyl)-1-piperazinyl]-2-methyl-4-pyrimidinyl]amino]-5-thiazolecarboxamide; and any other combination or dosing regimen comprising ′N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein, may be warranted. Alternatively, combinations of ′N-(2-chloro-6-methylphenyl)-2-[[6-[4-(2-hydroxyethyl)-1-piperazinyl]-2-methyl-4-pyrimidinyl]amino]-5-thiazolecarboxamide with a T315I inhibitor may also be warranted.

Therefore, the present invention provides a method of treating an individual suffering from a protein tyrosine kinase-associated disorder (where such individual is naïve to treatment with a kinase inhibitor (i.e., has not previously been treated with such) or has been treated with one or more kinase inhibitors (for example, has been treated with imatinib)), such as a SRC associated disorder (for example, a SRC-associated cancer), comprising: (a) providing a biological sample from said individual (whether as-is or manipulated (such as lysed), for example, to facilitate assaying); (b) assaying said biological sample for the presence of one or more mutant SRC kinase(s); and, based on the results of said assay, such as where one or more mutant SRC kinase(s) is (are) present in said sample, then (c) administering to said individual ′N-(2-chloro-6-methylphenyl)-2-[[6-[4-(2-hydroxyethyl)-1-piperazinyl]-2-methyl-4-pyrimidinyl]amino]-5-thiazolecarboxamide, preferably as an active agent in a pharmaceutical composition at a dose and/or frequency of dosing selected based on said assay (e.g., based on the sensitivity of such mutant(s) relative to with wild-type SRC kinase), and/or in combination with another protein tyrosine kinase inhibitor, including without limitation imatinib or AMN107, a tubulin stabilizing agent (e.g., such as pacitaxol, epothilone, taxone, etc.) a farnysyl transferase inhibitor such as COMPOUND II, and/or with another agent suitable for the treatment of said protein tyrosine kinase-associated disorder disclosed herein, said other kinase inhibitor and/or other agent being administered simultaneously or sequentially with the administration of ′N-(2-chloro-6-methylphenyl)-2-[[6-[4-(2-hydroxyethyl)-1-piperazinyl]-2-methyl-4-pyrimidinyl]amino]-5-thiazolecarboxamide, wherein, for example, and without limitation:

(1) identification of at least one mutant SRC kinase which is at least partially sensitive to inhibition with ′N-(2-chloro-6-methylphenyl)-2-[[6-[4-(2-hydroxyethyl)-1-piperazinyl]-2-methyl-4-pyrimidinyl]amino]-5-thiazolecarboxamide may optionally be used to select treatment with a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib preferentially to either kinase inhibitor alone or to other kinase inhibitor(s) (for example, where it is expected that the combination will be effective against said mutant at therapeutically useful doses better tolerated by patients than doses either kinase inhibitor alone or of such other kinase inhibitor(s));

(2) identification of at least one mutant SRC kinase may optionally be used to select the dose of the combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib, including increasing or decreasing the dose(s) of the individual agents thereof and/or the frequency of the dosing regimen (either for individuals naïve to ′N-(2-chloro-6-methylphenyl)-2-[[6-[4-(2-hydroxyethyl)-1-piperazinyl]-2-methyl-4-pyrimidinyl]amino]-5-thiazolecarboxamide or those undergoing treatment with ′N-(2-chloro-6-methylphenyl)-2-[[6-[4-(2-hydroxyethyl)-1-piperazinyl]-2-methyl-4-pyrimidinyl]amino]-5-thiazolecarboxamide), for example, where the mutant BCR/ABL kinase is inhibited by ′N-(2-chloro-6-methylphenyl)-2-[[6-[4-(2-hydroxyethyl)-1-piperazinyl]-2-methyl-4-pyrimidinyl]amino]-5-thiazolecarboxamide and/or imatinib to a lesser or greater degree, respectively, relative to WT SRC kinase; and/or

(3) identification of at least one mutant SRC kinase may optionally be used to select co-administration of another agent suitable for treatment of said protein tyrosine kinase-associated disorder in combination with ′N-(2-chloro-6-methylphenyl)-2-[[6-[4-(2-hydroxyethyl)-1-piperazinyl]-2-methyl-4-pyrimidinyl]amino]-5-thiazolecarboxamide, including and without limitation, a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., COMPOUND II); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor, for example, an inhibitor of BCR-ABL, such as imatinib and/or AMN-107; any other combination disclosed herein; and/or identification of at least one mutant SRC kinase with a sensitivity to ′N-(2-chloro-6-methylphenyl)-2-[[6-[4-(2-hydroxyethyl)-1-piperazinyl]-2-methyl-4-pyrimidinyl]amino]-5-thiazolecarboxamide decreased relative to other forms of such SRC kinase (e.g., WT SRC kinase) may optionally be used to select a dosing regimen of ′N-(2-chloro-6-methylphenyl)-2-[[6-[4-(2-hydroxyethyl)-1-piperazinyl]-2-methyl-4-pyrimidinyl]amino]-5-thiazolecarboxamide with an increased dose or increased dosing frequency regimen of ′N-(2-chloro-6-methylphenyl)-2-[[6-[4-(1-hydroxyethyl)-1-piperazinyl]-2-methyl-4-pyrimidinyl]amino]-5-thiazolecarboxamide relative to a treatment regimen suitable for such other forms of such SRC kinase (e.g., WT); and any other combination or dosing regimen comprising ′N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein, for example, where such agent is at least partially effective in inhibiting said mutant SRC kinase.

For use in the diagnostic and therapeutic applications described or suggested above, kits are also provided by the invention. Such kits may comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be an antibody or polynucleotide specific for a mutant SRC kinase protein or a mutant SRC kinase gene or message, respectively. Where the kit utilizes nucleic acid hybridization to detect the target nucleic acid, the kit may also have containers containing nucleotide(s) for amplification of the target nucleic acid sequence and/or a container comprising a reporter-means, such as a biotin-binding protein, such as avidin or streptavidin, bound to a reporter molecule, such as an enzymatic, florescent, or radioisotope label.

The kit of the invention will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific therapy or non-therapeutic application, and may also indicate directions for either in vivo or in vitro use, such as those described above.

Kits useful in practicing therapeutic methods disclosed herein may also contain a compound that is capable of inhibiting a mutant SRC kinase. Specifically contemplated by the invention is a kit comprising a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., COMPOUND II); a combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor, such as and without limitation, imatinib and/or AMN107; an increased dose and/or dosing frequency regimen of ′N-(2-chloro-6-methylphenyl)-2-[[6-[4-(2-hydroxyethyl)-1-piperazinyl]-2-methyl-4-pyrimidinyl]amino]-5-thiazolecarboxamide relative to relative to a treatment regimen suitable for such other forms of such SRC kinase (e.g., WT); and any other combination or dosing regimen comprising ′N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein, useful in treating mammals suffering from a mutant SRC associated disorder. For example, kits useful in identifying a mutant SRC kinase in a mammalian patient (e.g., a human) suffering from a cancer that is completely or partially resistant to, or has developed complete or partial resistance to, imatinib or another protein tyrosine kinase inhibitor and where said kits also comprise a therapeutically effective amount of the combination or increased dose or dosing regimen, are contemplated herein.

The following representative examples contain important additional information, exemplification and guidance which can be adapted to the practice of this invention in its various embodiments and the equivalents thereof. These examples are intended to help illustrate the invention, and are not intended to, nor should they be construed to, limit its scope.

### EXAMPLES

### Example 1

### Methods for Detecting Src Kinase Mutations

′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib are two potent SRC kinase inhibitors that are effective in treating CML and solid tumors. Provided herein are combination therapies and dosing regimens that will be useful in treating cancers which are resistant to protein tyrosine kinase inhibitor agents, such as imatinib and other kinase inhibitors, and specifically including cancers involving one or more mutations in SRC kinase.

A significant aspect of this combination therapy is the detection of the mutations in SRC kinase. If a mutant SRC kinase of the present invention is present in a patient, it indicates an individual can be selected for combination therapy, or more aggressive dosing regimens (e.g., higher and/or more frequent doses), or a combination of aggressive dosing regimen and combination therapy. Furthermore, if a specific SRC kinase mutant is detected, the amount of either or both inhibitors can be increased or decreased in order to enhance the therapeutic effect of the regimen.

There are several methods that can be used to detect a mutant SRC kinase in cancer patients, particularly CML patients. They include methods for detecting SRC kinase polynucleotides and SRC kinase proteins, as well as methods for identifying cells that express SRC kinase. Detection of a mutant SRC kinase in a patient would be diagnostic that such patients either are or will become at least partially resistant to imatinib therapy. As discussed in detail below, the status of SRC kinase gene products in patient samples may be analyzed by a variety of protocols well known in the art including, for example, immunohistochemical analysis, the variety of Northern blotting techniques including in situ hybridization, RT-PCR analysis (for example on laser capture micro-dissected samples), western blot analysis, tissue array analysis, microarray analysis, genotyping methods, and mass-spectroscopic methods.

Methods of identifying the nucleic acid and the amino acid of a mutant SRC kinase are known in the art.

One experimental strategy is to use PCR to amplify a region of the BCR-ABL transcript using primers specific to BCR and ABL, subclone this product and sequence at least 10 independent clones in both directions. This strategy allows one to quantify fluctuations in different clones from the same patient over time. Typical methodologies are for such protocols are provided below.

Blood samples can be obtained from patients enrolled in clinical trials in the treatment of CML. RNA is then extracted using TriAgent or TriAzol. cDNA synthesis is performed using MMTV reverse transcriptase. Polymerase chain reaction (PCR) is performed to amplify the cDNA, using primers CM10 (5′-GAAGCTTCTCCCTGACATCCGT-3′) (SEQ ID NO: 3) and 3′ Abl KD (5′-GCCAGGCTCTCGGGTGCAGTCC-3′) (SEQ ID NO: 4). A second round of PCR is performed to isolate the kinase domain using primers 5′ Abl KD, (5′-GCGCAACAAGCCCACTGTCTATGG-3′) (SEQ ID NO: 5) and 3′ Abl KD. The resultant 0.6 Kb fragment is then ligated into pBluescript II KS+digested with Eco RV. Bacterial transformants are plated on media containing ampicillin and X-gal. Ten white colonies per cDNA are inoculated into media and miniprep DNA is isolated. Sequencing of each clone is then performed using M13 universal forward (CGCCAGGGTTTTCCCAGTCACGAC; SEQ ID NO:5) and M13 reverse (AGCGGATAACAATTTCACACAGGA; SEQ ID NO:6) primers. Because two rounds of amplification will be employed, a mutation was considered present if it was detected on both strands of at least two independent clones per patient.

Alternatively, antibodies that immunospecifically bind to mutant SRC kinase may be used to detect the presence of a mutant SRC kinase in a sample. First, mutant SRC kinase may be generated by site directed mutagenesis. Cell lines expressing these mutant SRC kinase isoforms will then be created. Next, antibodies against mutant SRC kinase isoforms will be produced. Expression of SRC kinase and its mutant isoforms will be documented by Western blot analysis.

Specifically, site directed mutagenesis may be used to create the SRC kinase mutations (QuickChange Kit, Stratagene, La Jolla, Calif.) and all mutations will be confirmed by bidirectional sequencing (O'Farrell, A. M., et al., Blood, 101:3597-3605 (2003)). Retroviral transduction is performed and Ba/F3 cell lines stably expressing mutant SRC kinase isoforms are generated by double selection for G418 resistance and IL-3 independent growth (Yee, K. W., et al., Blood, 100:2941-2949 (2002); Yee, K. W., et al., Blood, 104:4202-4209 (2004); Tse, K. F., et al., Leukemia, 14:1766-1776 (2000); Schittenhelm, M. M., et al., manuscript submitted (2005)). Transient transfections of CHO-K1 chinese hamster cell lines with SRC kinase wild type (“WT”) or mutant isoforms are performed using a lipofection-assay (LipofectAMINE-kit purchased from Gibco-Invitrogen, Carlsbad, Calif.). Cells are treated with ′N-(2-chloro-6-methylphenyl)-2-[[6-[4-(2-hydroxyethyl)-1-piperazinyl]-2-methyl-4-pyrimidinyl]amino]-5-thiazolecarboxamide 24 hours after transfection (Heinrich, M. C., et al., Journal of Clinical Oncology, 21:4342-4349 (2003)). Alternatively, cells may be treated with any of the combinations outlined herein, or using increased levels of ′N-(2-chloro-6-methylphenyl)-2-[[6-[4-(2-hydroxyethyl)-1-piperazinyl]-2-methyl-4-pyrimidinyl]amino]-5-thiazolecarboxamide.

An anti-SRC kinase rabbit polyclonal antibody, an anti-STAT3 mouse monoclonal antibody (both Santa Cruz Biotechnology, Santa Cruz, Calif.), an anti-AKT (polyclonal) rabbit antibody (Cell Signaling Technology, Beverly Mass.) and an anti-MAP kinase 1/2 (Erk 1/2) rabbit monoclonal antibody (Upstate Biotechnology, Lake Placid, N.Y.) may be used at a 1:5000 to 1:2000 dilution. Anti-phosphotyrosine SRC antibodies (Tyr568/570 and Tyr703), an anti-phosphothreonine/tyrosine MAP kinase (Thr202/Tyr204) antibody, an anti-phosphothreonine (Thr308) and an anti-phosphoserine (Ser473) AKT antibody, an anti-phosphotyrosine (Tyr705) STAT3 antibody and an unspecific anti-phosphotyrosine antibody (clone pY20) are used at dilutions of 1:100 to 1:2000 (all Cell Signaling Technology, Beverly Mass.). Peroxidase conjugated goat anti-mouse antibody and goat anti-rabbit antibody will be used at 1:5000 and 1:10,000 dilutions respectively (BioRad; Hercules, Calif.). Protein A/G PLUS-Agarose immunoprecipitation reagent shall be purchased from Santa Cruz Biotechnology (Santa Cruz, Calif.). imatinib, ′N-(2-chloro-6-methylphenyl)-2-[[6-[4-(2-hydroxyethyl)-1-piperazinyl]-2-methyl-4-pyrimidinyl]amino]-5-thiazolecarboxamide, paclia tubulin stabilizing agent (e.g., pacitaxol, epothilone, taxane, etc.), and another agents useful in combination with ′N-(2-chloro-6-methylphenyl)-2-[[6-[4-(2-hydroxyethyl)-1-piperazinyl]-2-methyl-4-pyrimidinyl]amino]-5-thiazolecarboxamide, are dissolved in DMSO to create 10 mM stock solutions and be stored at −20° C.

Western blot assays may be conducted as follows. ˜5×10⁷ cells are exposed to varying concentrations of ′N-(2-chloro-6-methylphenyl)-2-[[6-[4-(2-hydroxyethyl)-1-piperazinyl]-2-methyl-4-pyrimidinyl]amino]-5-thiazolecarboxamide and cultured for 90 minutes at 37° C. in a 5% CO₂ atmosphere. Cell pellets are lysed with 100-150 μL of protein lysis buffer (50 mM Tris, 150 mM NaCl, 1% NP-40, 0.25% deoxycholate with added inhibitors aprotinin, AEBSF, leupeptin, pepstatin, sodium orthovanadate, and sodium pyruvate). 500-2000 microgram of protein from cell lysates are used for immunoprecipitation experiments and 75-200 microgram of protein from cell lysates are used for whole cell protein analysis by western immunoblot assays as previously described in Hoatlin, M. E., et al., Blood, 91:1418-1425 (1998).

In certain contexts, it may be desirable to amplify a specific region in SRC kinase such as one of the functional domains discussed herein. For example, the region corresponding to the ATP binding pocket and the activation loop domain of SRC is critical to the selectivity of imatinib and is the region known to harbor the most imatinib-resistant and protein tyrosine kinase inhibitor mutations. Sequencing of this region may most efficiently reveal the patients' CML clinical profile, and hence the appropriate combination therapy and/or dosing regimen. Briefly, RNA is extracted from purified peripheral blood or bone marrow cells with TriReagent-LS (Molecular Research Center, Inc., Cincinnati, Ohio). Total RNA is subjected to RT-PCR by using the same protocol and primers as described supra. PCR products are cloned into the pCR2.1 TA cloning vector (Invitrogen, Carlsbad, Calif.). Both strands of a 579-bp region are sequenced with the 5′ ABL primer and M13 forward primer or M13 forward and reverse primer set for the 1327-bp and the 579-bp fragments, respectively, on an ABI prism 377 automated DNA sequencer (PE Applied Biosystems, Foster City, Calif.). Sequence analysis is then performed using the ClustalW alignment algorithm). Any detected mutation is then confirmed by analysis of genomic DNA. Briefly, genomic DNA is extracted from purified bone marrow or peripheral blood cells with the QiaAMP Blood Mini Kit (Qiagen, Inc., Valencia, Calif.). A 361-bp DNA fragment is amplified by PCR with two primers (5′-GCAGAGTCAGAATCCTTCAG-3′ (SEQ ID NO: 7) and 5′-TTTGTAAAAGGCTGCCCGGC-3′) (SEQ ID NO: 8) which are specific for intron sequences 5′ and 3′ of ABL exon 3, respectively. PCR products are cloned and sequenced.

Additional methods of detecting mutant SRC kinases is disclosed in O'Hare et al. (Cancer Research, 65(11):4500-5 (2005), which is hereby incorporated by reference in its entirety).

### Example 2

### Method of Assessing the Potential of the Combination Therapy

The combination of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib may be studied in mouse models of imatinib-resistant or protein tyrosine kinase inhibitor resistant, SRC-dependent disease. A series of such pharmacodynamic experiments will help to determine the optimal dosing regimen for different mutant SRC isoforms in vivo. Pharmacodynamic experiments are well known in the art and one skilled in the art would readily appreciate that such experiments can be modified to alter existing conditions, as applicable. Briefly, severe combined immuno-deficient mice are injected intravenously with Ba/F3 cells expressing different SRC wild-type or mutant isoforms as well as the firefly luciferase gene. Untreated mice harboring Ba/F3 cells expressing nonmutant or imatinib-resistant SRC mutants are expected to develop aggressive disease, with massive liver and splenic infiltration, typically resulting in death. To assess the ability of combination therapy, or a modified dosing regimen, to inhibit SRC in vivo, SRC kinase activity in splenocyte lysates prepared at various time points after administration of a different single dose of 0, 0.5, 1, 5, and 10 micromoles per liter of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib combination by oral gavage will be assessed. Phosphorylation of the adapter protein CRKL, a known BCR-ABL substrate (T. Oda et al., J. Biol. Chem. 269, 22925 (1994)), will be monitored to gauge the efficacy of the combination therapy. On the basis of a series of such pharmacodynamic experiments, an proper dose of the combination will be chosen for efficacy studies. Then, mice will be documented by bioluminescence imaging before and after dosing. On the basis of a series of such pharmacodynamic experiments, the optimal dosing regimen and/or combination therapy may be identified. Mice are dosed with combination or vehicle alone by gavage for 2 weeks, beginning 3 days after injection of Ba/F3 cells, and disease burden is then assessed by bioluminescence imaging. All vehicle-treated mice are expected to develop progressive disease. In contrast, combination-treated mice harboring nonmutant SRC or the clinically common imatinib-resistant and protein tyrosine kinase inhibitor resistant mutations described herein are expected to develop less or no progressive disease. It is also expected that different optimal dosing regimens will be identified for different SRC isoforms. Such dosing difference may be taken into consideration in the treatment of patients with a known SRC mutation(s).

### Example 3

### Method of Assessing the Safety and Efficacy of Protein Tyrosine Kinase Combination Therapy and/or Modified Dosing Regimens

Previous findings have shown that both ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib are highly selective for leukemic versus normal hematopoietic cells (B. J. Druker et al., Nature Med. 2. 561 (1996) and N. P. Shah et al., Science 305. 399 (2004)). Such high selectivity demonstrates the high safety and efficacy of these inhibitors, and the expected efficacy of their combination. To assess the efficacy of the combination on human bone marrow progenitors, the compounds are tested in vitro in colony-forming-unit (CFU) assays. A series of concentrations of ′N-(2-chloro-6-methylphenyl)-2-[[6-[4-(2-hydroxyethyl)-1-piperazinyl]-2-methyl-4-pyrimidinyl]amino]-5-thiazolecarboxamide and imatinib combination, or other combinations disclosed herein, are applied to bone marrow progenitors isolated from healthy volunteers and from CML patients with either imatinib-sensitive (nonmutant SRC) or imatinib-resistant disease. Furthermore, the blast-forming unit-erythroid (BFU-E) and CFU-granulocyte-monocyte (GM) colonies from CML patient marrow samples will be analyzed by polymerase chain reaction (PCR) analysis in order to detect the sensitivity of selection for growth of rare normal progenitors present in these leukemic marrow samples. Briefly, bone marrow is harvested from clinical subjects. Viable frozen Ficoll-Hypaque-purified mononuclear cells are thawed and grown overnight in Iscove's Media supplemented with 10% Fetal calf serum, 1-glutamine, pen-strep, and stem cell factor (100 ug/ml) at a density of 5×10⁵/ml. After 24 hours, viable cells are quantitated and plated in Methocult media (Cell Signal Technologies, Beverly, Mass.) at 1×10⁴ and 1×10⁵ cells per plate in the presence of 5 nM ′N-(2-chloro-6-methylphenyl)-2-[[6-[4-(2-hydroxyethyl)-1-piperazinyl]-2-methyl-4-pyrimidinyl]amino]-5-thiazolecarboxamide or vehicle. Experiments are performed in triplicate. On day 11, erythroid blast-forming unit (BFU-E) and granulocyte-macrophage colony forming units (CFU-GM) will be quantitated. On day 14, colonies will be isolated with a pipet tip, and RNA will be isolated using a Qiagen Rneasy kit. A primer complementary to the region of ABL approximately 200 nucleotides downstream of the BCR/ABL mRNA (5′-CGGCATTGCGGGACACAGGCCCATGGTACC; SEQ ID NO:9) junction is annealed to purified RNA. cDNA is synthesized using mouse Moloney leukemia virus (MMLV) reverse transcriptase, and subjecting to 40 cycles of PRC using either a BCR (5′-TGACCAACTCGTGTGTGAAACT; SEQ ID NO:10) or ABL type Ia 5′ primer (GGGGAATTCGCCACCATGTTGGAGATCTGCCTGA; SEQ ID NO: 11) as a control for the quality of RNA.

### Example 4

### Methods for Measuring of Bcr-Abl Kinase Activity via the phosphotyrosine content of Crkl

The ability of a combination therapy or more aggressive dosing regimen of the present invention to effectively overcome imatinib resistance, to inhibit BCR-ABL activity, or to inhibit BCR-ABL mutant activity, the phosphotyrosine content of Crkl, an adaptor protein which is specifically and constitutively phosphorylated by Bcr-Abl in CML cells may be used (see, e.g. J. ten Hoeve et al., Blood 84, 1731 (1994); T. Oda et al., J. Biol. Chem. 269, 22925 (1994); and G. L. Nichols et al., Blood 84, 2912 (1994)). The phosphotyrosine content of Crkl has been shown to be reproducibly and quantitatively measured in clinical specimens. Crkl binds Bcr-Abl directly and plays a functional role in Bcr-Abl transformation by linking the kinase signal to downstream effector pathways (see, e.g. K. Senechal et al., J. Bio. Chem. 271, 23255 (1996)). When phosphorylated, Crk1 migrates with altered mobility in SDS-PAGE gels and can be quantified using densitometry. Sawyers et al (U.S. Ser. No. 10/171,889, filed Jun. 16, 2002; incorporated herein by reference) have shown that Crk1 phosphorylation in primary CML patient cells was inhibited in a dose-dependent manner when exposed to STI-571 and correlated with dephosphorylation of Bcr-Abl. Likewise, we have also shown that Crk1 phosphorylation was inhibited in a dose-dependent manner when exposed to ′N-(2-chloro-6-methylphenyl)-2-[[6-[4-(2-hydroxyethyl)-1-piperazinyl]-2-methyl-4-pyrimidinyl]amino]-5-thiazolecarboxamide (data not shown). Thus, such a Crk1 assay will allows for an assessment of the enzymatic activity of Bcr-Abl protein in a reproducible, quantitative fashion and be a useful means of assessing the ability of a combination therapy or more aggressive dosing regimen of the present invention to effectively overcome imatinib resistance, to inhibit BCR-ABL activity, or to inhibit BCR-ABL mutant activity.

Briefly, cells are lysed in 1% Triton X-100 buffer with protease and phosphatase inhibitors (see, e.g. A. Goga et al., Cell 82, 981 (1995)). Equal amounts of protein, as determined by the BioRad DC protein assay (Bio-RadLaboratories, Hercules, Calif.), are separated by SDS-PAGE, transferred to nitrocellulose and immunoblotted with phosphotyrosine antibody (4G10, Upstate Biotechnologies, Lake Placid, N.Y.), Abl antibody (pex5, (see, e.g. A. Goga et al., Cell 82, 981 (1995)), β-actin antibody (Sigma Chemicals, St. Louis, Mo.) or Crk1 antiserum (Santa Cruz Biotechnology, Santa Cruz, Calif.). Immunoreactive bands are visualized by ECL (Amersham Pharmacia Biotech, Piscataway, N.J.). Several exposures are obtained to ensure linear range of signal intensity. Optimal exposures are quantified by densitometry using ImageQuant software (Molecular Dynamics, Sunnyvale, Calif.)).

### Example 5

### Methods for Examining Amplification of the BCR-ABL Gene in Mammalian Cells

An additional method of assessing the ability of a combination therapy or more aggressive dosing regimen of the present invention to effectively overcome imatinib resistance, to inhibit BCR-ABL activity, or to inhibit BCR-ABL mutant activity is provided. Specifically, dual-color fluorescence in situ hybridization (FISH) experiments may be performed to determine if BCR-ABL gene amplification is effectively diminished. The latter is based upon the appreciation in the art that BCR-ABL amplification is observed in imatinib-resistant and protein tyrosine kinase inhibitor resistant patients. Briefly, interphase and metaphase cells are prepared (see, e.g. E. Abruzzese et al, Cancer Genet. Cytogenet. 105, 164 (1998)) and examined using Locus Specific Identifier (LSI) BCR-ABL dual color translocation probe (Lysis, Inc., Downers Grove, Ill.)). Cytogenetic and FISH characterization of metaphase spreads may be observed to assess if an inverted duplicate Ph-chromosome with interstitial amplification of the BCR-ABL fusion gene is present.

Alternatively, quantitative PCR analysis of genomic DNA obtained from patients may be used to assess if BCR-ABL gene amplification is present. Briefly, genomic DNA may be extracted from purified bone marrow or peripheral blood cells with the QiaAMP Blood Mini Kit (Qiagen, Inc., Valencia, Calif.). 10 ng of total genomic DNA is subjected to real-time PCR analysis with the iCycler iQ system (Bio-Rad Laboratories, Hercules, Calif.). A 361-bp cDNA fragment including ABL exon 3 is amplified using two primers (5′-GCAGAGTCAGAATCCTTCAG-3′ (SEQ ID NO: 7) and 5′-TTTGTAAAAGGCTGCCCGGC-3′ (SEQ ID NO: 8)) which are specific for intron sequences 5′ and 3′ of ABL exon 3, respectively. A 472-bp cDNA fragment of glyceraldehyde-3-phosphate dehydrogenase (GAPDH) is amplified using two primers (5′-TTCACCACCATGGAGAAGGC-3′ (SEQ ID NO: 12) and 5′-CAGGAAATGAGCTTGACAAA-3′ (SEQ ID NO: 13)) which are specific for sequences in exon 5 and exon 8 of GAPDH, respectively. Fold increase in ABL copy number may be determined by calculating the difference between threshold cycle numbers of ABL and GAPDH for each sample (DCt). A control may be used as a reference sample, DCt from each sample may be subtracted from DCt of control to determine D(DCt). Fold increase is then calculated as 2^{-D(DCt)}.

### Example 6

### Art Accepted Methods for Measuring Enzymological and Biological Properties of BCR-ABL Mutants

A variety of assays for measuring the enzymological properties of protein kinases such as Abl are known in the art, for example those described in Konopka et al., Mol Cell Biol. November 1985; 5(11):3116-23; Davis et al., Mol Cell Biol., January 1985; 5(1):204-13; and Konopka et al., Cell. Jul. 1, 1984; 37(3):1035-42 the contents of which are incorporated herein by reference. Using such assays the skilled artisan can measure the enzymological properties of mutant BCR-Abl protein kinases and to assess the ability of a combination therapy or more aggressive dosing regimen of the present invention to effectively overcome imatinib resistance, to inhibit BCR-ABL activity, or to inhibit BCR-ABL mutant activity.

A variety of bioassays for measuring the transforming activities of protein kinases such as Abl are known in the art, for example those described in Lugo et al., Science. Mar. 2, 1990; 247(4946):1079-82; Lugo et al., Mol Cell Biol. March 1989; 9(3):1263-70; Klucher et al., Blood. May 15, 1998; 91(10):3927-34; Renshaw et al., Mol Cell Biol. March 1995; 15(3):1286-93; Sitard et al., Blood. Mar. 15, 1994; 83(6):1575-85; Laneuville et al., Cancer Res. Mar. 1, 1994; 54(5):1360-6; Laneuville et al., Blood. Oct. 1, 1992; 80(7):1788-97; Mandanas et al., Leukemia. August 1992; 6(8):796-800; and Laneuville et al., Oncogene. February 1991; 6(2):275-82 the contents of which are incorporated herein by reference. Using such assays the skilled artisan can measure the phenotype of mutant BCR-Abl protein kinases.

Additional methods are disclosed in O'Hare et al. (Cancer Research, 65(11):4500-5 (2005), which is hereby incorporated by reference in its entirety.

The entire disclosure of each document cited (including patents, patent applications, journal articles, abstracts, laboratory manuals, books, Genbank Accession numbers, SWISS-PROT Accession numbers, or other disclosures) in the Background of the Invention, Detailed Description, Brief Description of the Figures, and Examples is hereby incorporated herein by reference in their entirety. Further, the hard copy of the Sequence Listing submitted herewith, in addition to its corresponding Computer Readable Form, are incorporated herein by reference in their entireties.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

What is claimed is:

1. A method for determining the responsiveness of an individual with a protein tyrosine kinase-associated disorder to treatment with ′N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt or hydrate thereof, wherein said individual has either been previously treated with and developed at least partial resistance to a first kinase inhibitor, or is naïve to treatment with kinase inhibitors, comprising: (a) providing a biological sample from said individual; (b) screening said biological sample for the presence of at least one mutation in a BCR/ABL kinase sequence; wherein the presence of said at least one mutation is indicative of the patient requiring either treatment with said thiazolecarboxamide or a pharmaceutically acceptable salt or hydrate thereof; a higher dose or dosing frequency of said thiazolecarboxamide or a pharmaceutically acceptable salt or hydrate thereof; or combinations of said thiazolecarboxamide or a pharmaceutically acceptable salt or hydrate thereof with a tubulin stabilizing agent, a farnysyl transferase inhibitor, a BCR/ABL T315I inhibitor and/or another protein tyrosine kinase inhibitor; or both a higher dose or dosing frequency of said thiazolecarboxamide or a pharmaceutically acceptable salt or hydrate thereof in addition to said one or more combinations.

2. The method of claim 1 wherein said mutant BCR/ABL kinase comprises an amino acid mutation that results in said BCR/ABL kinase being constitutively active.

3. The method of claim 1 wherein said protein tyrosine kinase-associated disorder is selected from the group consisting of chronic myeloid leukemia (CML), Ph+ ALL, AML, imatinib-resistant CML, and solid tumors.

4. The method of claim 1 wherein said mutant BCR/ABL kinase comprises an imatinib resistant BCR/ABL mutation or a protein tyrosine kinase inhibitor resistant mutation.

5. The method of claim 4 wherein said imatinib resistant BCR/ABL mutation or protein tyrosine kinase inhibitor resistant mutation comprises a mutation at amino acid positions 244, 250, 252, 253, 255, 315, 317, 351, 355, 359, 396 and 486 of SEQ ID NO:2.

6. The method of claim 5 wherein said mutation is selected from the group consisting of M244V, G250E, Q252H, Q252R, Y253F, Y253H, E255K, E255V, T315I, F317L, M351T, E355G, F359V H396R, and F486S of SEQ ID NO:2.

7. The method of claim 1, wherein said biological sample is selected from the group consisting of a tissue biopsy, blood, serum, plasma, lymph, ascitic fluid, cystic fluid, urine, sputum, stool, saliva, bronchial aspirate, spinal fluid and hair.

8. The method of claim 7 wherein said biological sample is a tissue biopsy cell sample or cells cultured therefrom.

9. The method of claim 7 wherein said biological sample comprises a member of the group consisting of: (a) blood cells; (b) cells removed from a solid tumor; and (c) a lysate of the cell sample of (a) or (b).

10. A method of treating an individual suffering from a protein tyrosine kinase associated disorder comprising: (a) providing a biological sample from said individual; (b) assaying said biological sample for the presence of a mutant BCR/ABL kinase; and if a mutant BCR/ABL kinase is identified (c) administering a therapeutically effective amount of ′N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt or hydrate thereof, alone or in combination with imatinib.

11. The method of claim 10 wherein said mutant BCR/ABL kinase comprises a mutation at amino acid positions 244, 250, 252, 253, 255, 315, 317, 351, 355, 359, 396 and 486 of BCR/ABL kinase (SEQ ID NO:2).

12. The method of claim 11 wherein said mutation is selected from the group consisting of M244V, G250E, Q252H, Q252R, Y253F, Y253H, E255K, E255V, T315I, F317L, M351T, E355G, F359V H396R, and F486S of SEQ ID NO:2.

13. A method of treating an individual suffering from a protein tyrosine kinase associated disorder comprising: (a) providing a biological sample from said individual; (b) assaying said biological sample for at least partial resistance to a first kinase inhibitor; (c) assaying said biological sample for the presence of a mutant BCR/ABL kinase; and if said biological sample is determined to be at least partially resistant to said first kinase inhibitor and contains a mutant BCR/ABL kinase, then (d) administering a therapeutically effective amount of ′N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt or hydrate thereof; a higher dose or dosing frequency of said thiazolecarboxamide or a pharmaceutically acceptable salt or hydrate thereof; or combinations of said thiazolecarboxamide or a pharmaceutically acceptable salt or hydrate thereof with a tubulin stabilizing agent, a farnysyl transferase inhibitor, a BCR/ABL T315I inhibitor and/or another protein tyrosine kinase inhibitor; or both a higher dose or dosing frequency of said thiazolecarboxamide or a pharmaceutically acceptable salt or hydrate thereof in addition to said one or more combinations.

14. The method of claim 13 wherein said first kinase inhibitor is a BCR/ABL kinase inhibitor.

15. The method of claim 13 wherein said mutant BCR/ABL kinase comprises a mutation at amino acid positions 244, 250, 252, 253, 255, 315, 317, 351, 355, 359, 396 and 486 of SEQ ID NO:2.

16. The method of claim 15 wherein said mutation is selected from the group consisting of M244V, G250E, Q252H, Q252R, Y253F, Y253H, E255K, E255V, T315I, F317L, M351T, E355G, F359V H396R, and F486S of SEQ ID NO:2.

17. The method of claim 13 wherein said first kinase inhibitor comprises imatinib.

18. A method of establishing a treatment regimen for an individual suffering from a protein tyrosine kinase associated disorder comprising: (a) providing a biological sample from said individual; (b) screening said biological sample for the presence of at least one mutation in a BCR/ABL kinase sequence; and, if at least one mutation in a BCR/ABL kinase sequence is present in said biological sample, (c) administering to said individual as part of a treatment regimen a pharmaceutical composition comprising ′N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt or hydrate thereof; a higher dose or dosing frequency of said thiazolecarboxamide or a pharmaceutically acceptable salt or hydrate thereof; or combinations of said thiazolecarboxamide or a pharmaceutically acceptable salt or hydrate thereof with a tubulin stabilizing agent, a farnysyl transferase inhibitor, a BCR/ABL T315I inhibitor and/or another protein tyrosine kinase inhibitor; or both a higher dose or dosing frequency of said thiazolecarboxamide or a pharmaceutically acceptable salt or hydrate thereof in addition to said one or more combinations.

19. The method of claim 18 wherein said at least on mutation comprises a mutation at amino acid positions 244, 250, 252, 253, 255, 315, 317, 351, 355, 359, 396 and 486 of SEQ ID NO:2.

20. The method of claim 19 wherein said pharmaceutical composition comprises at least one additional kinase inhibitor.

21. The method of claim 20 wherein said mutation is selected from the group consisting of M244V, G250E, Q252H, Q252R, Y253F, Y253H, E255K, E255V, T315I, F317L, M351T, E355G, F359V H396R, and F486S.

22. A method of establishing a treatment regimen for an individual suffering from a protein tyrosine kinase associated disorder comprising: (a) providing a biological sample from said individual; (b) assaying said biological sample for at least partial resistance to a first kinase inhibitor; (c) assaying said biological sample for the presence of at least one mutation in a BCR/ABL kinase sequence; and, if said biological sample is determined to be at least partially resistant to said first kinase inhibitor and contain a mutant BCR/ABL kinase, then (d) administering to said individual as part of a treatment regimen a pharmaceutical composition comprising ′N-(2-chloro-6-methylphenyl)-2-[[6-[4-(2-hydroxyethyl)-1-piperazinyl]-2-methyl-4-pyrimidinyl]amino]-5-thiazolecarboxamide, a higher dose or dosing frequency of said thiazolecarboxamide or a pharmaceutically acceptable salt or hydrate thereof; or combinations of said thiazolecarboxamide or a pharmaceutically acceptable salt or hydrate thereof with a tubulin stabilizing agent, a farnysyl transferase inhibitor, a BCR/ABL T315I inhibitor and/or another protein tyrosine kinase inhibitor; or both a higher dose or dosing frequency of said thiazolecarboxamide or a pharmaceutically acceptable salt or hydrate thereof in addition to said one or more combinations.

23. A method of treating an individual suffering from a mutant BCR/ABL kinase associated disorder comprising: (a) providing a biological sample from said individual; (b) assaying said biological sample for the presence of a mutant BCR/ABL kinase, wherein said mutant BCR/ABL kinase is constitutively active; and, if a constitutively active mutant BCR/ABL kinase is present in said sample, then (c) administering to said individual a pharmaceutical composition comprising ′N-(2-chloro-6-methylphenyl)-2-[[6-[4-(2-hydroxyethyl)-1-piperazinyl]-2-methyl-4-pyrimidinyl]amino]-5-thiazolecarboxamide, a higher dose or dosing frequency of said thiazolecarboxamide or a pharmaceutically acceptable salt or hydrate thereof; or combinations of said thiazolecarboxamide or a pharmaceutically acceptable salt or hydrate thereof with a tubulin stabilizing agent, a farnysyl transferase inhibitor, a BCR/ABL T315I inhibitor and/or another protein tyrosine kinase inhibitor; or both a higher dose or dosing frequency of said thiazolecarboxamide or a pharmaceutically acceptable salt or hydrate thereof in addition to said one or more combinations.

24. A method of treating an individual suffering from a mutant BCR/ABL kinase associated disorder comprising administering a therapeutically effective amount of ′N-(2-chloro-6-methylphenyl)-2-[[6-[4-(2-hydroxyethyl)-1-piperazinyl]-2-methyl-4-pyrimidinyl]amino]-5-thiazolecarboxamide, a higher dose or dosing frequency of said thiazolecarboxamide or a pharmaceutically acceptable salt or hydrate thereof; or combinations of said thiazolecarboxamide or a pharmaceutically acceptable salt or hydrate thereof with a tubulin stabilizing agent, a farnysyl transferase inhibitor, a BCR/ABL T315I inhibitor and/or another protein tyrosine kinase inhibitor; or both a higher dose or dosing frequency of said thiazolecarboxamide or a pharmaceutically acceptable salt or hydrate thereof in addition to said one or more combinations.

25. A kit for use in determining treatment strategy for an individual with a protein tyrosine kinase-associated disorder, comprising: (a) a means for detecting a mutant BCR/ABL kinase in a biological sample from said patient; and optionally (b) instructions for use and interpretation of the kit results.

26. The kit according to claim 25, wherein said treatment strategy comprises administration of a therapeutically effective amount of ′N-(2-chloro-6-methylphenyl)-2-[[6-[4-(2-hydroxyethyl)-1 piperazinyl]-2-methyl-4-pyrimidinyl]amino]-5-thiazolecarboxamide, or a pharmaceutically acceptable salt or hydrate thereof, a higher dose or dosing frequency of said thiazolecarboxamide or a pharmaceutically acceptable salt or hydrate thereof; or combinations of said thiazolecarboxamide or a pharmaceutically acceptable salt or hydrate thereof with a tubulin stabilizing agent, a farnysyl transferase inhibitor, a BCR/ABL T315I inhibitor and/or another protein tyrosine kinase inhibitor; or both a higher dose or dosing frequency of said thiazolecarboxamide or a pharmaceutically acceptable salt or hydrate thereof in addition to said one or more combinations.

27. A kit for use in treating an individual with a mutant BCR/ABL kinase associated disorder, comprising: (a) a means for detecting mutations at amino acid positions 244, 250, 252, 253, 255, 315, 317, 351, 355, 359, 396 and 486 of a BCR/ABL kinase from a biological sample from said individual; (b) a therapeutically effective amount of ′N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt or hydrate thereof; a higher dose or dosing frequency of said thiazolecarboxamide or a pharmaceutically acceptable salt or hydrate thereof; or combinations of said thiazolecarboxamide or a pharmaceutically acceptable salt or hydrate thereof with a tubulin stabilizing agent, a farnysyl transferase inhibitor, a BCR/ABL T315I inhibitor and/or another protein tyrosine kinase inhibitor; or both a higher dose or dosing frequency of said thiazolecarboxamide or a pharmaceutically
